(19) United States
(12) Patent Application Publication
(10) Pub. No.: US 2004/0220130 A1
(43) Pub. Date: Nov. 4, 2004

(54) Compact synthetic expression vector comprising double-stranded DNA molecules and methods of use thereof

(76) Inventors: Paul D. Robbins, Mt. Lebanon, PA (US); Jeffrey C. Mai, Pittsburgh, PA (US)

Correspondence Address: BAKER & BOTTS, 30 ROCKEFELLER PLAZA, NEW YORK, NY, 10112

(21) Appl. No.: 10/807,755

(22) Filed: Mar. 24, 2004

Related U.S. Application Data
(60) Provisional application No. 60/456,989, filed on Mar. 24, 2003.

(51) Int. Cl. A61K 48/00; C07H 21/04

(52) U.S. Cl. 514/44; 536/23.2; 435/320.1

(57) ABSTRACT

The present invention relates to a safe, simple, compact synthetic and potentially non-toxic vector that is comprised of two or more complementary strands of chemically-synthesized deoxyribonucleic (DNA) acid that, when annealed to one another, form a cassette for the efficient expression of single-stranded or double-stranded ribonucleic acid (RNA) molecules. The RNA molecules expressed from this synthetic vector may function as ribozymes, as antisense molecules, or as short, interfering RNA molecules. Alternatively, the RNA molecules may encode therapeutic or antigenic peptides, polypeptides, or proteins. This synthetic vector may be employed for the rapid screening of various candidate RNA molecules for their efficacy in gene silencing or other functions, or for the delivery of RNA molecules in therapeutic applications.

### CROSS REFERENCE TO RELATED APPLICATIONS

This application is claims benefit of U.S. Provisional Patent Application Serial No. 60/456,989, filed Mar. 24, 2003, the contents of which are incorporated by reference herein in its entirety.

### STATEMENT REGARDING FEDERALLY SPONSORED RESEARCH

The subject matter described herein was supported in part by National Institutes of Health Grant so that the United States Government has certain rights herein.

### INTRODUCTION

The present invention relates to a synthetic vector. The vector is safe, simple, potentially non-toxic and compact. The invention also relates to methods of using this vector to express oligonucleotides in mammalian target cells or tissues. Such oligonucleotides include RNA molecules, and particularly short RNA molecules capable of producing gene silencing by RNA interference, translational repression, splice suppression, or ribozyme-mediated degradation of mRNA. The vector of the instant invention is useful, inter alia, for the rapid screening of various candidate RNA molecules for their efficacy in gene silencing, and in the delivery of RNA molecules in therapeutic applications.

### BACKGROUND OF INVENTION

Existing Vectors and their Limitations

A number of viral and nonviral delivery systems have been developed, including vectors derived from human adenoviruses, herpes simplex viruses, adeno-associated viruses (Mulligan, Science 1993;260:926-932; Berns and Giraud, Ann. N.Y. Acad. Sci. 1995;772:95-104; Smith, Ann. Rev. Microbiol. 1995;49:807-838) and a host of others. The cell recognition specificity of viruses and the vectors derived therefrom is generally very high and their ability to transfer genetic material into a target cell makes them particularly attractive candidates for the delivery of genetic material to a target cell. However, there are potential risks and limitations associated with the use of viral vectors for the delivery of genetic material, including the possibility of insertional mutagenesis with integrating vectors such as retroviral vectors, and adverse host reactions against other viral vectors such as adenovirus or the cells transduced by these vectors. Targeting of specific cell types, the need for replication of the targeted cell for efficient expression of the genetic material, and the production of viral vectors in titers sufficient for in vivo applications are also significant problems. See e.g. Yang et al., J. Virol. 1995;69:2004-2015.

Nonviral delivery systems include so-called naked nucleic acids and nucleic acids complexed or conjugated to lipids or proteins. These vectors provide several fundamental advantages over viral vectors, including simplicity of design, ease of production, superiority of yield, and batch-to-batch reproducibility. For many of the nonviral vectors, especially naked nucleic acids, the toxic or immunogenic properties associated with viral vectors may be minimized simply through the total elimination of lipid or protein components. However, even with the complete elimination of other biological materials, DNA itself has been shown to be immunogenic under certain conditions. This immunogenicity is thought to be caused largely by the presence of undermethylated CpG dinucleotides, which are a consequence of replication of the nucleic acid in bacteria. While such reactions may be avoided through the use products of the polymerase chain reaction (PCR) rather than plasmids propagated in bacteria, the yield of PCR products is low and PCR reactions contain enzymes, lipids and other components that may still be a source of contamination.

Thus, there is a continuing need for the development of new vectors that are safe and non-toxic, simple to design, inexpensive to make, and that may be tested rapidly in both non-clinical and clinical settings.

Potential Therapeutic Uses of Ribonucleic Acids (RNAs)

One potential application for vectors is the delivery of RNA into a target cell. Over the past two decades, a growing number of studies have demonstrated potential therapeutic applications for RNAs. See Opalinska and Gewirtz, Nat Rev Drug Discov 2002;1:503-14. Perhaps the first potential therapeutic use recognized for an RNA molecule was as a means for inhibiting translation of a specific messenger RNA (mRNA), thereby inhibiting the expression of the protein encoded by the mRNA. This inhibition is accomplished through the formation of a double-stranded RNA molecule, which is not accessible to the cellular protein translation machinery, following the introduction into the cell of a RNA that is complementary (or ‘antisense’) to the target mRNA. Although promising in theory, a large number of difficulties exist in the art surrounding antisense technology. Most commonly, efficient synthesis of an exogenous ssRNA antisense molecule and its delivery to the target cell are difficult to achieve.

Another form of RNA with potential therapeutic uses is the ribozyme, which is a RNA molecule that catalytically cleaves RNA in a sequence specific manner, resulting in the degradation of the RNA and consequently a reduction in the amount of protein translated from the RNA. Zaug et al., Nature 1986;324:429-33. The use of ribozymes as potential gene regulators in mammalian cells and antiviral agents has been suggested. However, because ribozymes are ssRNA molecules and thus similar to antisense RNA molecules, the synthesis and delivery problems encountered in applying antisense technology to disease treatment are also encountered in the use of ribozyme technology.

Double-stranded RNA (dsRNA) molecules also may be therapeutically useful. For example, early reports indicated that dsRNAs are important in the induction of interferon synthesis, implicating virally-derived dsRNA molecules in the initiation of interferon-mediated anti-viral immune responses (for a review, see Jacobs and Langland, Virology 1996;219:339-349). In addition, dsRNAs have been reported to have anti-proliferative properties (Hubbell et al., Proc. Natl. Acad. Sci. USA 1991;88:906910); synthetic dsRNAs have been shown to inhibit tumor growth in mice (Levy et al., Proc. Nat. Acad. Sci. USA 1969;62:357-361), to be active in the treatment of leukemic mice (Zeleznick et al., Proc. Soc. Exp. Biol. Med. 1969;130:126-128), and to inhibit chemically-induced tumorigenesis in mouse skin (Gelboin et al., Science 1970; 167:205-207).

More recently, a role for dsRNA has been observed in silencing gene expression. First observed in Caenorhabditis elegans (Lee et al., Cell 1993;75:843-54; Reinhart et al., Nature 2000;403:901-906), this process of RNA interference is triggered by certain forms of dsRNA. Introduction of the dsRNA into cells expressing the appropriate molecular machinery leads to degradation of the corresponding endogenous mRNA. The mechanism involves conversion of dsRNA into short RNAs that direct ribonucleases to homologous mRNA targets (for a review, see Ruvkun, Science 2001;2294:797-799). This process is related to normal defense against viruses and the mobilization of transposons.

As shown by several recent reports, RNAi provides a rapid method to test the function of genes. Most of the genes on chromosome Is and III of the nematode Caenorhabditis elegans now have been tested for RNAi phenotypes. See Tavernarakis, Nat. Genet. 2000;24:180-183; Barstead, Curr. Opin. Chem. Biol. 2001;5:63-66; Zamore, Nat. Struct. Biol. 2001;8:746-750. However, when used in vertebrate species, RNAi initially was found to be unpredictable, operating with very low efficiencies. Fjose et al., Biotechnol. Annu. Rev. 2001;7:31-57. For example, when tested in zebrafish embryos, RNAi was proven not to be a viable technique for studying gene function (Zhao et al., Dev. Biol. 2001;229:215-223), yet it was effective when used in Xenopus embryos (Nakano et al., Biochem. Biophys. Res. Commun. 2000;274:434-439). Furthermore, Svoboda et al. reported that RNAi provides a suitable and robust approach to study the function of dormant maternal mRNAs in mouse oocytes. Svoboda et al., Development 2000;127:4147-4156. These inconsistent observations may reflect the notorious difficulties in the synthesis of dsRNA and its efficient delivery into target cells.

In light of the foregoing examples of potential investigational and therapeutic applications of functional ss and ds RNA molecules, it is clear that a need remains in the art for a reliable and effective method for safe, simple, and efficient expression of ss and ds RNA molecules in various mammalian target cells and tissues. The present invention addresses this need by providing a synthetic vector that is useful, inter alia, for the efficient intercellular expression of ss and ds RNA molecules.

### SUMMARY OF THE INVENTION

The present invention relates to a synthetic vector. As used herein, “synthetic” means made wholly by chemical means, e.g. through the annealing of chemically-synthesized complementary oligonucleotides rather than by biological means, e.g. through the amplification of a chemically-synthesized template using the polymerase chain reaction (PCR) or other enzyme-mediated biological reactions such as ligation or phosphorylation. The synthetic vector of the instant invention is safe, simple, compact, and is preferably non-toxic. In one embodiment, it is comprised of two or more complementary strands of deoxyribonucleic acid (DNA). When annealed to one another, the two or more complementary strands of DNA form a cassette which is useful for the efficient expression of single-stranded (ss) or double-stranded (ds) RNA molecules that may function as ribozymes, as antisense molecules, as short, interfering RNA (siRNA) molecules for RNA interference, or in any other function associated with RNA molecules of approximately 0-90 base pairs (bp) in length. ds DNA molecules may also be expressed from the cassette contained within the synthetic vector of the instant invention. The vector may be either linear or circular.

The synthetic vector of the instant invention is the most compact expression vector for gene therapy yet known and the only vector that can be synthesized wholly by chemical means. Moreover, the complete lack of viral proteins or lipid elements drastically minimizes possible adverse immunological reactions against the vector or the vector-transduced cells. This vector represents a significant advance over other vectors currently employed for ex vivo and in vivo gene therapy, and can be used for the rapid screening of various candidate RNA molecules for their efficacy in gene silencing, or for the delivery of RNA molecules in therapeutic applications.

### DESCRIPTION OF THE FIGURES

The present invention may be better understood with reference to the attached figures, in which

FIG. 1 is a schematic diagram depicting a strategy for the construction of one exemplary embodiment of the compact synthetic vector of the instant invention;

FIGS. 2A-B depict the primary nucleic acid sequence (SEQ ID NO:1) and secondary structure of silencing hairpin RNA molecule directed against the humanized, enhanced Green Fluorescent Protein (heGFP). Panel A shows the structure of the RNA molecule before processing by RNase III. Panel B shows the structure of the RNA molecule after processing by RNase III (SEQ ID NO:2 and SEQ ID NO:3);

FIG. 3 shows the sequence of the sense strand of the compact expression vector (SEQ ID NO:4), wherein bold, underlined sequence corresponds to the human H1 RNA Promoter (Pol III), italicized sequence corresponds to the EGFP silencing hairpin transcript comprising 20 bp of complementarity, the underlined, italicized sequence corresponds to the RNA loop of the hairpin transcript, and the non-bold, underlined sequence corresponds to the terminator sequence for Pol III;

FIGS. 4A-H: Panel A shows a phase-contrast photomicrograph of HEK 293 cells transfected with 500 ng of the H1-eGFP construct. Panel B shows a fluorescence photomicrograph of HEK 293 cells transfected with 500 ng of the H1-eGFP construct. Panel C shows a phase-contrast photomicrograph of HEK 293 cells transfected with 500 ng of peGFP-Luc construct. Panel D shows a fluorescence photomicrograph of HEK 293 cells transfected with 500 ng of the peGFP-Luc construct. Panel E shows a phase-contrast photomicrograph of HEK 293 cells transfected with 500 ng of the peGFP-Luc construct and 200 ng of the H1-eGFP construct. Panel F shows a fluorescence photomicrograph of HEK 293 cells transfected with 500 ng of the peGFP-Luc construct and 200 ng of the H1-eGFP construct. Panel G shows a phase-contrast photomicrograph of HEK 293 cells transfected with 500 ng of the peGFP-Luc construct and 500 ng of the H1-eGFP construct, and H shows a fluorescence photomicrograph of HEK 293 cells transfected with 500 ng of the peGFP-Luc construct and 500 ng of the H1-eGFP construct;

FIG. 5 shows eGFP expression in HEK 293 cells quantified by flow cytometry;

FIG. 6 is a schematic diagram illustrating one exemplary method for incorporating a protein ligand into the compact synthetic vector to facilitate targeting and delivery;

FIG. 7 confirms the efficacy of coupling of protein ligands to nucleic acid molecules via a 3′ Amino C3 group, wherein lane 1 shows starting, uncoupled RNA, lanes 2 and 4 show coupled Ac-6R-RNA, and lanes 3 and 5 show coupled 6CF-6R-RNA;

FIG. 8 depicts two alternatives strategies for the construction of the compact synthetic vector that do not require the use of PCR;

FIG. 9 is a schematic diagram depicting a potential configuration of the compact synthetic vector containing a truncated Pol III promoter;

FIG. 10 is a schematic diagram depicting a potential configuration of the compact synthetic vector containing a partial hairpin in which only the antisense strand is present;

FIG. 11 is a schematic diagram depicting a potential configuration of the compact synthetic vector in which the type 1 Pol III promoter regulates production of the primary transcript;

FIGS. 12A-B are schematic diagrams depicting exemplary embodiments of the compact synthetic vector in which the type 2 Pol III promoter regulates production of the primary transcript, which may be either a complete siRNA or other functional RNA (panel A) or a partial siRNA hairpin (panel B);

FIG. 13 is a schematic diagram depicting an exemplary embodiment of the compact synthetic vector containing core elements of the Pol II promoter;

FIGS. 14A-B are schematic diagrams depicting exemplary embodiments of the compact synthetic vector in which a Pol II core promoter based on the adenovirus-2 major late promoter (AdML2) regulates production of the primary transcript, which may be either a complete siRNA or other functional RNA (panel A) or a partial siRNA hairpin (panel B);

FIGS. 15A-B are schematic diagrams depicting exemplary embodiments of the compact synthetic vector in which a Pol II core promoter based on the glial cell-specific human papovavirus JC core promoter (JCV) regulates production of the primary transcript, which may be either a complete siRNA or other functional RNA (panel A) or a partial siRNA hairpin (panel B);

FIG. 16 is a schematic diagram depicting an exemplary embodiment of the compact synthetic vector containing a tethered artificial transcription factor to enhance initiation of transcription from a Pol II core promoter;

FIG. 17 is a schematic diagram depicting an exemplary embodiment of the compact synthetic vector containing a additional elements enabling induction of transcription from Pol II or Pol III promoters;

FIG. 18 is a schematic diagram depicting the induction of transcription from the compact synthetic vector depicted in FIG. 17 following removal of 17β-estradiol;

FIG. 19 is a schematic diagram depicting an exemplary embodiment of the compact synthetic vector in which a variant type 3 Pol III promoter regulates production of the primary transcript;

FIG. 20 is a schematic diagram depicting the compact synthetic vector configured as a ‘micro-circle;’

FIG. 21 is a schematic diagram depicting a ‘micro-circular’ configuration of the compact synthetic vector that contains a replication origin;

FIG. 22 is a schematic diagram depicting a strategy for the synthesis and validation of the compact synthetic vector of depicted in FIG. 21;

FIGS. 23A-C depict the primary nucleic acid sequence (Panel A; SEQ ID NO:14 and SEQ ID NO:15) of compact synthetic vector for the production of a primary (Panel B; SEQ ID NO:16) and mature (Panel C; SEQ ID NO:17 and SEQ ID NO:18) silencing hairpin RNA molecule directed against the β-catenin 1 gene;

FIGS. 24A-B depict a schematic diagram of a compact synthetic vector (Panel A) that employs the tRNA Valine promoter to regulate the expression of a functional RNA molecule (Panel B) that is ultimately processed by RNase III to generate a siRNA molecule specific for eGFP;

FIGS. 25A-B depict a schematic diagram of a compact synthetic vector (Panel A) that employs the human 87U6 internal promoter to regulate the expression of a functional RNA molecule (Panel B) that is ultimately processed by RNase III to generate a siRNA molecule specific for eGFP;

FIG. 26 is a schematic diagram of a compact synthetic vector that incorporates the HSH1 promoter to regulate the expression of a siRNA molecule;

FIG. 27 is a schematic diagram of a compact synthetic vector that incorporates the adenovirus major late gene promoter to regulate the expression of a siRNA molecule;

FIG. 28 is a schematic diagram of a compact synthetic vector that incorporates the JCV promoter to regulate the expression of a siRNA molecule;

FIG. 29 is a schematic diagram of a compact synthetic vector that incorporates the HSH1 promoter to regulate the expression of an antisense RNA molecule;

FIGS. 30A-H: Panel A shows a fluorescence photomicrograph of HEK 293 cells 48 hr after transfection with a negative control plasmid (pUC19). Panel B shows a fluorescence photomicrograph of HEK 293 cells 48 hr after transfection with 200 ng of an eGFP plasmid. Panel C shows a fluorescence photomicrograph of HEK 293 cells 48 hr after transfection with 200 ng of the eGFP expression plasmid and 200 ng of a plasmid containing the “IA” REC. Panel D shows a fluorescence photomicrograph of HEK 293 cells 48 hr after transfection with 200 ng of the eGFP expression plasmid and 500 ng of a plasmid containing the “IA” REC. Panel E shows a fluorescence photomicrograph of HEK 293 cells 48 hr after transfection with 200 ng of the eGFP expression plasmid and 1000 ng of a plasmid containing the “IA” REC. Panel F shows a fluorescence photomicrograph of HEK 293 cells 48 hr after transfection with 200 ng of the eGFP expression plasmid and 1000 ng of a plasmid containing the “JA” REC. Panel shows a fluorescence photomicrograph of HEK 293 cells 48 hr after transfection with 500 ng of the eGFP expression plasmid and 1000 ng of a plasmid containing the “JA” REC. Panel H shows a fluorescence photomicrograph of HEK 293 cells 48 hr after transfection with 1000 ng of the eGFP expression plasmid and 1000 ng of a plasmid containing the “JA” REC; and

FIGS. 31A-H: Panel A shows a fluorescence photomicrograph of HEK 293 cells 72 hr after transfection with a negative control plasmid (pUC19). Panel B shows a fluorescence photomicrograph of HEK 293 cells 72 hr after transfection with 200 ng of an eGFP plasmid. Panel C shows a fluorescence photomicrograph of HEK 293 cells 72 hr after transfection with 200 ng of the eGFP expression plasmid and 200 ng of a plasmid containing the “IA” REC. Panel D shows a fluorescence photomicrograph of HEK 293 cells 72 hr after transfection with 200 ng of the eGFP expression plasmid and 500 ng of a plasmid containing the “IA” REC. Panel E shows a fluorescence photomicrograph of HEK 293 cells 72 hr after transfection with 200 ng of the eGFP expression plasmid and 1000 ng of a plasmid containing the “IA” REC. Panel F shows a fluorescence photomicrograph of HEK 293 cells 72 hr after transfection with 200 ng of the eGFP expression plasmid and 1000 ng of a plasmid containing the “JA” REC. Panel shows a fluorescence photomicrograph of HEK 293 cells 72 hr after transfection with 500 ng of the eGFP expression plasmid and 1000 ng of a plasmid containing the “JA” REC. Panel H shows a fluorescence photomicrograph of HEK 293 cells 72 hr after transfection with 1000 ng of the eGFP expression plasmid and 1000 ng of a plasmid containing the “JA” REC is based on the human H1 promoter system.

### DETAILED DESCRIPTION OF THE INVENTION

As reviewed above, functional ss and ds RNA molecules are of great interest in both investigational and therapeutic settings, provided that adequate amounts of these molecules could be delivered efficiently and inexpensively into the target cell. The synthetic vector of the instant invention provides an inexpensive method for the safe, simple, and efficient expression of ss and ds RNA molecules in various mammalian target cells and tissues. The synthetic vector of the instant invention system further provides for the expression of ds DNA molecules, and consequently for the intracellular expression of therapeutic and/or antigenic peptides encoded by ds DNA molecules.

As used herein, “synthetic” means made wholly by chemical means, e.g. through the annealing of chemically-synthesized complementary oligonucleotides rather than by biological means, e.g. through the amplification of a chemically-synthesized template using the polymerase chain reaction (PCR) or other enzyme-mediated biological reactions such as ligation or phosphorylation. In preferred embodiments, the oligonucleotides from which the vector is formed are synthesized using commercial oligonucleotide synthesis machines, including but not limited to the ABI 394 and ABI 3900 DNA/RNA Synthesizers available from Applied Biosystems, Inc. or other commercially-equivalent synthesizers.

The use of the term “synthetic” herein thus may be at odds with the meaning of this phrase as sometimes employed in the scientific or technical literature, wherein a “synthetic vector” sometimes has been construed to mean a plasmid isolated following its creation and propagation in prokaryotic cells, or a gene delivery system comprising such a plasmid in combination with lipids, proteins or other biological materials. Such vectors are not the synthetic vectors of the instant invention.

In accordance with the present invention, a synthetic vector is provided. This vector is safe, simple, and compact, and is preferably non-toxic. In a preferred embodiment, the synthetic vector comprises two or more complementary strands of deoxyribonucleic acid (DNA). When annealed to one another, the two or more complementary strands of DNA form a cassette for the efficient expression of single-stranded (ss) or double-stranded (ds) RNA molecules or ds DNA molecules. The RNA molecules may function as ribozymes, as antisense molecules, as short, interfering RNA (siRNA) molecules for RNA interference, or in any other function associated with ss or ds RNA molecules of approximately 0-90 base pairs (bp) in length. ds DNA molecules may also be expressed from the cassette contained within the synthetic vector of the instant invention. The vectors formed from the annealed oligonucleotides may be either linear or circular ds DNA molecules.

In an exemplary embodiment, the invention comprises a synthetic vector for the expression of RNA that comprises two or more complementary oligonucleotides. When annealed, the oligonucleotides form a double-stranded DNA expression cassette having a first region that regulates RNA transcription, i.e., a promoter, a second region from which the functional ss or ds RNA molecule is transcribed, and a third region that terminates the transcription.

In a preferred embodiment, the synthetic vector is less than 135 bp in length and comprises two complementary oligonucleotides. In alternative embodiments where the synthetic vector is greater than 135 bp in length, it comprises more than two complementary oligonucleotides. When the vector comprises more than two oligonucleotides, the oligonucleotides may be configured so that there is preferably at least twelve base pairs of overlap, and more preferably at least about 20-50 base pairs of overlap, between the oligonucleotides constituting the opposite strands of the vector. In certain embodiments, the synthetic vector may be from about 50 bp to about 2000 bp in length. The overall length of the oligonucleotides comprising the vector is limited by current DNA synthesis technology at about 135 bp, but the present invention also encompasses longer oligonucleotides provided that they may be synthesized chemically.

The synthetic vector may be either a linear or a circular ds DNA molecule. In preferred embodiments, the vector is a linear molecule of less than 135 bp in length. In these linear forms, targeting peptides or other moieties may be incorporated into the 5′ ends of the oligonucleotides comprising each of the complementary strands of the vector via 1-ethyl-3-(3-dimethylaminopropyl)-carbodiimide (EDC)-mediated coupling to an amino-C6 group present on the 5′ end of each oligonucleotide or through other bifunctional crosslinking agents capable of stably or reversibly linking the DNA with a chosen moiety. Preferred moieties for incorporation into the vector include protein transduction domains (PTDs), RGD peptides (peptides containing Arg-Gly-Asp motifs), a receptor ligand such as folate, antibodies, nuclear localization sequences (NLSs), endosmolytic peptides, etc. Alternative moieties to be incorporated into the 5′ end of the oligonucleotide include fluorescent beacons including, but not limited to, the dye Cy3.

In its circular embodiments, the synthetic vector of the instant invention further contains, in addition to the promoter region, the transcribed region and the transcription termination region, one of the mammalian origins of replication known to those of skill in the art. An example of a mammalian replication origin suitable for use in the vector of the instant application is the 36 bp REPori A3/4 origin of replication of SEQ ID NO:19.

In certain embodiments, the promoter region may be the wild-type or a modified form of the human H1 polymerase III promoter, including preferably the approximately 100 bp human H1 RNA type 3 polymerase III promoters of SEQ ID NOS:20 and 21 or the approximately 70 bp human H1 RNA type 3 polymerase III promoter of SEQ ID NO:22. In alternative embodiments, the promoter region may be derived from the human type 1 Pol III promoter, the human type 2 Pol III promoter, a variant of the human type 3 Pol III promoter, or a Pol II promoter.

Specific embodiments of synthetic vectors incorporating a human type 2 Pol III promoter are set forth in SEQ ID NOS:23-28, wherein “N” indicates the presence of variable sequences that may be made specific for the sense and antisense strands of the targeted gene. Sense and antisense regions may be present in either order in the hairpin generating region of the transcript. The regions containing these variable sequences are 19 bp in length in these specific embodiments, but may vary in length between 15 bp and 30 bp. Embodiments in which the total length of the synthetic vector is greater than 135 bp can be generated through the annealing of more than two oligonucleotides, each of which is less than 135 bp in length.

Specific embodiments of synthetic vectors incorporating a variant human type 3 Pol III promoter are set forth in SEQ ID NOS:29-30. Again, “N” indicates the presence of variable sequences that may be made specific for the sense and antisense strands of the targeted gene, and sense and antisense regions may be present in either order in the hairpin generating region of the transcript. The regions containing these variable sequences are 19 bp in length in these specific embodiments, but may vary in length between 15 bp and 30 bp. Embodiments in which the total length of the synthetic vector is greater than 135 bp can be generated through the annealing of more than two oligonucleotides, each of which is less than 135 bp in length.

Specific embodiments of synthetic vectors incorporating the adenovirus major late promoter (AdML2) are set forth in SEQ ID NOS:31-32. As above, “N” indicates the presence of variable sequences that may be made specific for the sense and antisense strands of the targeted gene, and sense and antisense regions may be present in either order in the hairpin generating region of the transcript. The regions containing these variable sequences are 19 bp in length in these specific embodiments, but may vary in length between 15 bp and 30 bp. Embodiments in which the total length of the synthetic vector is greater than 135 bp can be generated through the annealing of more than two oligonucleotides, each of which is less than 135 bp in length.

The promoters to be employed in the instant invention also may be modified so that they display cell or tissue specificity. For example, the human pol II promoter may be engineered to achieve cell- or tissue-specific expression through the incorporation of minimal elements from tissue-specific promoters including, but not limited to, the promoters for the genes encoding prepro-endothelin-1, myelin basic protein, metallothionein, the neurofibromatosis-1 (NF1) protein, growth hormone factor 1 (GHF-1), peripherin, fibroin, JC virus (JCV) proteins, and the period-1 (PER1) protein. Each of these minimal promoters is sufficiently compact so that it readily may be incorporated into the vector of the instant invention. In this context, minimal elements of tissue-specific promoters are those regions of the promoter sequence that are required to maintain at least 10% of wild-type promoter activity in the cell or tissue type in which the wild-type promoter is normally expressed.

In a preferred embodiment, the tissue-specific promoter comprises the glial cell-specific promoter derived from the human papovavirus JC core promoter as set forth in SEQ ID NO:7. Specific embodiments of synthetic vectors incorporating the JCV minimal promoter elements are set forth in SEQ ID NOS:33-34. Again, “N” indicates the presence of variable sequences that may be made specific for the sense and antisense strands of the targeted gene, and sense and antisense regions may be present in either order in the hairpin generating region of the transcript. The regions containing these variable sequences are 19 bp in length in these specific embodiments, but may vary in length between 15 bp and 30 bp. Embodiments in which the total length of the synthetic vector is greater than 135 bp can be generated through the annealing of more than two oligonucleotides, each of which is less than 135 bp in length.

The promoters to be employed in the instant invention also may be modified so that their activity is inducible. For example, the human pol II promoter may be engineered to incorporate a binding site for factors that interact with elements upstream of the transcription start site. Examples of such factors include, but are not limited to, dexamethasone (glucocorticoid receptor), doxycycline (the “tet” system), 17β-estradiol (estrogen receptor), and ecdysone among others. In a preferred embodiment, the human pol II is engineered to incorporate the estrogen response elements A and B (SEQ ID NO:10 and SEQ ID NO:11, respectively).

In an additional embodiment, the Pol II promoter of the instant invention may be modified to incorporate its own tethered artificial transcription factor. This modification can aid in overcoming the potential competition of the pol II promoter of the vector with pol II promoters of the cell for the limited pool of pol II transcriptional machinery. In the case of the instant invention, this tethering may be done through succinimidyl-6-maleimidlyhexanoate (EMCS; Molecular Probes)-mediated linkage of a cysteine-containing transactivator peptide to an amine-modified nucleotide located at either the 3′ or the 5′ ends of one or more of the oligonucleotides comprising the vector. Other covalent linkages also may be possible.

The artificial transcription factors may be peptides derived from, among other proteins, the acidic domain of the viral protein VPI6. These peptides may be synthesized from the naturally-occurring “L” amino acids or the nonnaturally-occurring “D” forms for increased stability. In preferred embodiments, the artificial transcription factors are the acidic domain (AD) peptides AD-16 (CGSDALDDFDLDMLGS; SEQ ID NO:8) or AD-29 (CGSDALDDFDLDMLGSDALDDFDLDMLGS; SEQ ID NO:9).

The synthetic vectors of the instant invention may further comprise heteroduplex “bubbles” located between the promoter region and the transcriptional start site. Such heteroduplex bubbles may be four or more nucleotides in length, and are generated through the introduction of specific nucleotides into one strand of the vector that do not base pair with the nucleotides present at the corresponding positions of the complementary strand. The heteroduplex bubbles facilitate strand separation and thus potentiate promoter activity by up to 100-fold. In preferred embodiments utilizing Pol II promoters, the heteroduplex bubbles comprise the nucleotides spanning from positions −9 to +3, −9 to −1, or −14 to −3 relative to the start of transcription. In preferred embodiments utilizing Pol III promoters, the bubble comprises the nucleotides spanning from the −9 to the −5 positions or the nucleotides spanning from the +2 to the +6 positions. Such bubbles bypass the requirements for B″ protein or Brf protein in transcriptional activation, respectively. See Kassavetis et al., EMBO J. 2001;20:2823-2834.

Various heteroduplex regions for both Pol II and Pol III promoters based on these findings are possible. As described by Pan and Greenblatt (J. Biol. Chem. 1994;269:30101-30104) the heteroduplexes are designed such that the sequence of the non-transcribed strand is altered so that its base pairing with the transcribed strand is disrupted. In the instant invention, the designated heteroduplex region is comprised of the sequence of the transcribed strand (bottom strand) which is duplicated and replaces the corresponding region of the non-transcribed strand (top strand), but any nucleotide changes that disrupt basepairing in these regions are functionally equivalent to the designs recited herein. See Kassavetis et al., EMBO J. 2001;20:2823-2834.

The transcribed regions of the synthetic vectors of the instant invention may contain DNA sequences encoding ss or ds RNA molecules including, but not limited to, those that are functional in RNA interference or other forms of RNA silencing, translational repression, splice suppression, as antisense repressors of protein translation, or as ribozymes. In a preferred embodiment, the transcribed region of the synthetic vector of the instant invention encodes the ss RNA silencing hairpin of SEQ ID NO:1, which is specific for the humanized enhanced Green Fluorescent Protein gene. In an alternative embodiment, the transcribed region of the synthetic vector of the instant invention encodes the ss RNA silencing hairpin of SEQ ID NO:16, which is specific for the β-catenin gene. When expressed intracellularly, these ss RNA molecules can be converted into short, interfering RNA molecules by the actions of RNase III. Hairpin RNAs that give rise to short, interfering RNA molecules following intracellular expression may comprise both the sense and antisense strands or, alternatively, only the antisense strand specific for the targeted gene. In an alternative embodiment, the transcribed region of the synthetic vector of the instant invention may give rise to a ss RNA molecule that encodes therapeutic or antigenic peptides, polypeptides or proteins.

The synthetic vectors of the present invention may be modified through the incorporation of one or more phosphorothioate groups, non-natural bases, 5′ and 3′ overhangs, 5′ and 3′ modifications, and mismatches into one or both strands of the dsDNA to extend their biological half-life.

The present invention further provides a synthetic vector made by annealing two or more complementary synthetic oligonucleotides to form a double-stranded DNA molecule. When the synthetic vector comprises more than two oligonucleotides, such that each strand contains one or more breaks between two adjoining oligonucleotides, such breaks may be repaired using techniques known to those of ordinary skill in the art, such as by “filling in” or ligation reactions. See Ausubel, ed. Current Protocols in Molecular Biology. J. Wiley & Sons. 1993. Alternatively, such breaks may be repaired by transfecting the vector into a cell, wherein the breaks may be repaired intracellularly the cell's own DNA repair mechanisms provided that the oligonucleotides are 5′ phosphorylated. In preferred embodiments, intrastrand breaks are repaired intracellularly.

The present invention further provides a method for expressing functional ss or ds RNA molecules in a target cell comprising administering a vector to the target cell wherein the vector is comprised of two or more complementary synthetic oligonucleotides. The synthetic vectors may be introduced into the target cell by any standard technique, including transfection, transduction, electroporation, bioballistics, microinjection, etc. The vector may further comprise any number of modifications known to those of ordinary skill in the art to enhance cellular transduction. For example, the vector may be incorporated into a liposome, or be conjugated to peptides, lipids or other cellular ligands. In preferred embodiments, the vector is covalently conjugated to a protein transduction domain (PTD), an RGD peptide, a folate molecule, an antibody, a nuclear localization sequence (NLS), or an endosmolytic peptide. Administration may occur either in vitro or in vivo. For in vivo applications, the vector may be administered by intravenous injection, by intraperitoneal injection, parenterally, by direct injection to the target site, or by any other means known to those of ordinary skill in the art to result in delivery of the vector to the target cell. The instant invention further provides for compositions comprising the vector and a suitable carrier. Carriers suitable for the delivery of DNA vectors are known to those of ordinary skill in the art.

The present invention further provides a method of inhibiting gene expression comprising administering to a target cell a vector comprised of two or more complementary synthetic oligonucleotides, wherein the vector encodes a ss hairpin RNA molecule that can be converted intracellularly into a short, interfering RNA molecule through the actions of RNase III, an antisense oligonucleotide, or a ribozyme. In preferred embodiments, the vector expresses a ss hairpin RNA molecule as the means of inhibiting gene expression. The hairpin molecule may contain both sense and the antisense strands specific for the target gene or, preferably, only the antisense strand. In a preferred embodiment, the ss hairpin RNA molecule is the ss hairpin RNA of SEQ ID NO:1, which is specific for the humanized enhanced Green Fluorescent Protein gene. In another preferred embodiment, the ss hairpin RNA molecule is the ss hairpin RNA of SEQ ID NO:16, which is specific for the β-catenin gene.

The invention is further illustrated by the following examples, which are not intended to limit the scope of the invention.

### EXAMPLES

Materials and Methods

Vector Design and Construction. An H1-EGFP construct containing the human H1 Pol III promoter (positions −100 to −1) driving expression of a 20 bp RNA hairpin that is complementary to humanized eGFP was generated by PCR using two long, overlapping oligos (95 bases each; 34 bp overlap). Each oligo has an amino-C6 group added to the 5′-end to allow for coupling to COOH-containing ligands by EDC coupling. In some studies, each oligo was coupled to an Ac-6R peptide ligand. The final product, 156 bp long and either uncoupled or coupled to a peptide ligand, was purified on silica membrane columns and eluted in dH₂O.

Transfection Studies. Human 293 cells were co-transfected with the peGFPLuc vector (Clontech) expressing humanized eGFP and the H1-EGFP dsRNA cassette at varying concentrations (200-500 ng per well in a 24-well plate; cells at 40% confluency) using Effectene (Qiagen). Media (DMEM, 10% FCS) was left unchanged during the course of the experiment. Cells were incubated at 37C, 5% CO₂.

Fluorescence was examined in the samples at 24, 48 and 96 hours post-transfection. At 96 hours post-transfections, cells were collected and subjected to analysis by flow cytometry for net eGFP fluorescence.

RNA Shadowing. 5.1 μg of RNA was loaded onto each lane of an 8% polyacrylamide gel containing 7M urea. Samples were electrophoresed and RNA was then visualized by RNA shadowing, performed as described. See Olejnik et al., Nucl. Acids Res. 1998;26:3572-3576.

### Example 1

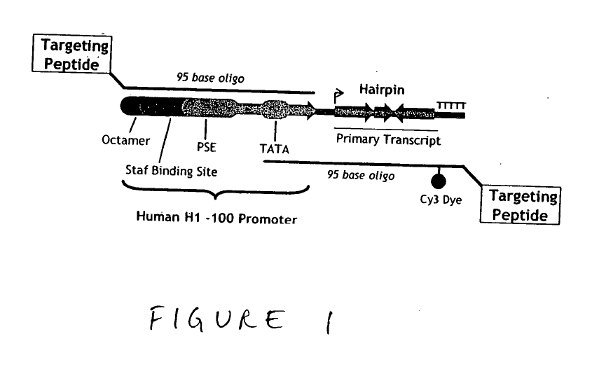

A Compact Vector Wherein Expression of a siRNA is Regulated by the Type 3 Pol III Promoter Can Be Generated by PCR. The polymerase chain reaction (PCR) was used to generate a double-stranded DNA vector capable of expressing a variety of useful RNA molecules. As shown schematically in FIG. 1, this highly compact vector is comprised of a minimal human H1 RNA type 3 polymerase III (Pol III) promoter and DNA sequences that encode the desired RNA molecule. The Pol III promoter region utilized in this vector consists of approximately 100 bp, and lacks many of the internal control elements normally present in the Pol III promoter region. This vector has a predicted molecular weight of approximately 110 kDa.

Although not the synthetic vector of the instant invention, this vector nevertheless was useful to establish the functionality of the basic design of the synthetic vector. To generate the approximately 160 bp vector, two oligonucleotides, each 95 bp in length with approximately 30-35 bp of overlap, representing complementary strands of the double-stranded DNA vector were synthesized chemically and used as a template for PCR. Chemical synthesis of the oligonucleotides used as primers for the PCR amplification permits many synthetic modifications to be incorporated into the resulting vector. For example, as indicated in FIG. 1, carboxyl-containing targeting peptides or other peptide ligands may be incorporated into the 5′ ends of these priming oligonucleotides via 1-ethyl-3-(3-dimethylaminopropyl)-carbodiimide (EDC)-mediated coupling to an amino-C6 group present on the 5′ end of each oligonucleotide. Potential ligands to be incorporated into the vector in this manner include protein transduction domains (PTDs), RGD peptides, folate, antibodies, nuclear localization sequences (NLSs), endosmolytic peptides, etc. Initial ligands to be coupled include the 6R PTD for protein transduction delivery and the RGD motif for integrin targeting. Fluorescent beacons such as the dye Cy3 may also be incorporated into the vector via attachment to the synthetic PCR priming oligonucleotides to assist in tracking the cellular uptake and intracellular distribution of the vector.

One potential RNA species that may be delivered by a vector such as the one detailed in FIG. 1 is a single-stranded RNA (ssRNA) hairpin that can be converted into a short, interfering RNA (siRNA) molecule when expressed intracellularly. One example of such a ssRNA hairpin is depicted in FIG. 2. Panel A of this figure shows the secondary structure of a the molecule. The stem is comprised of 20 bp of sense and antisense sequence that is specific for the humanized, enhanced Green Fluorescent Protein (heGFP; Clontech, Palo Alto Calif.). When this hairpin structure is expressed in a cell, RNase III digests the loop of the hairpin, generating the dsRNA molecule shown in Panel B of FIG. 2. It is important to note that the resulting molecule contains a 5′ phosphate and 3′ hydroxyl termini, and two single-stranded nucleotides (“UU”) on the 3′ ends of each strand. Such structural features are critical for the entry of the siRNA molecule into the RNAi pathway; blunt-ended siRNAs or siRNAs lacking a 5′ phosphate group elicit only weak responses in vitro and in vivo.

Other RNA molecules that may be expressed from vectors of the type illustrated in FIG. 1 include, but are not limited to, those that are functional in RNA silencing, translational repression, splice suppression, as antisense repressors of protein translation, and as ribozymes.

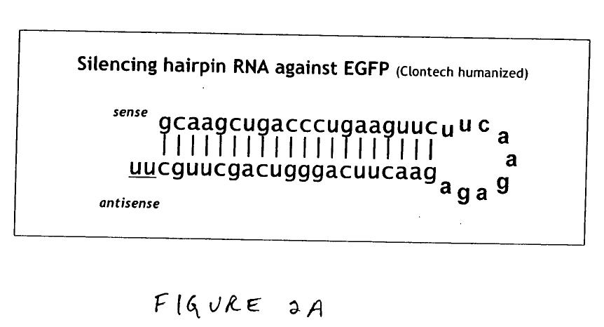

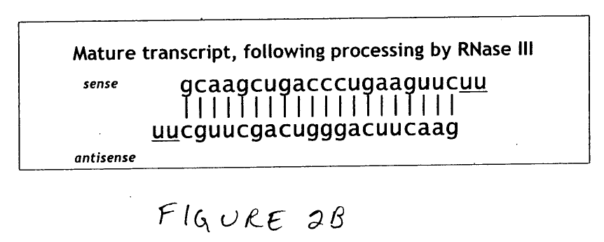

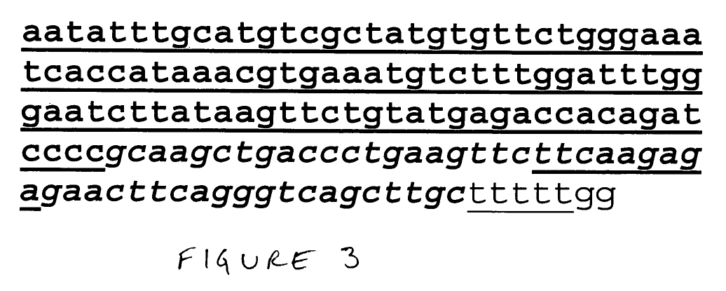

The complete 156 bp nucleotide sequence of the sense strand of the compact expression vector of FIG. 1 is shown in FIG. 3. The locations of the sequences encoding the human H1 RNA promoter, the eGFP silencing hairpin transcript, and the pol III terminator are indicated.

### Example 2

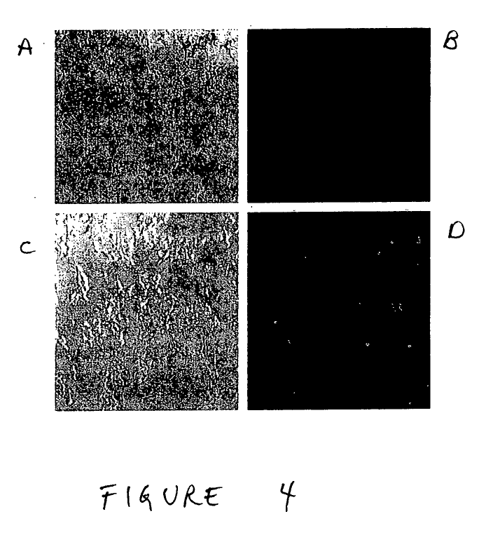

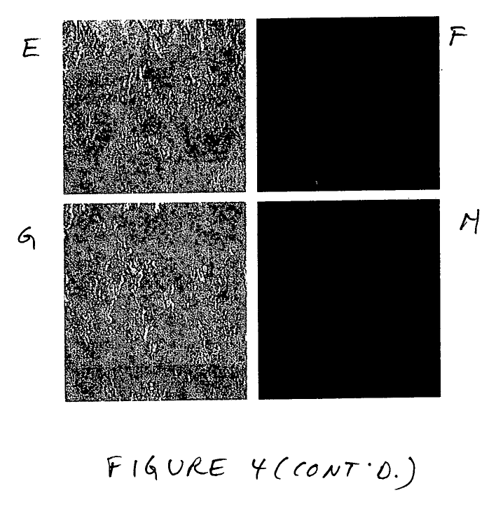

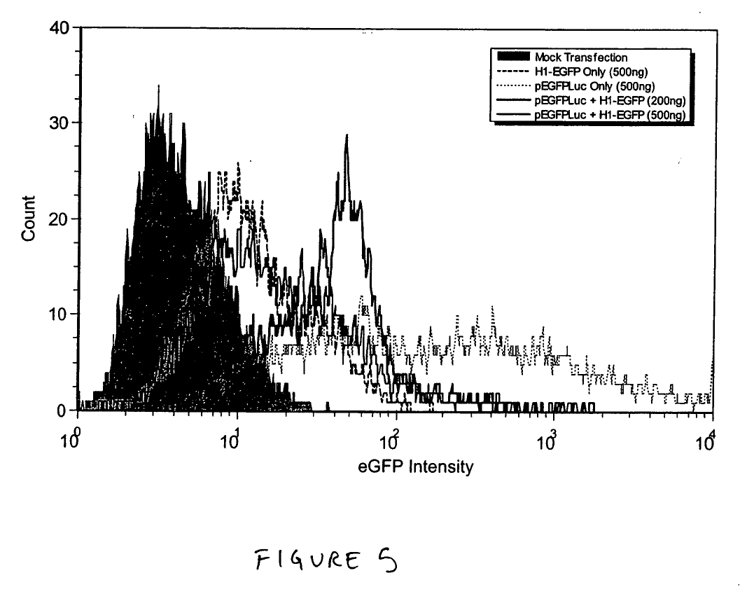

A Compact Vector Expressing a siRNA Specific for the Green Fluorescent Protein Gene Reduced Expression of this Protein in Cultured Human Cells. In vitro studies were performed to determined whether a siRNA generated from the compact vector described above could silence gene expression in human cells. In these studies, the results of which are summarized in FIG. 4 and FIG. 5, human 293 cells were transfected with either 1) 500 ng of the eGFP-specific siRNA-expressing synthetic vector (H1-eGFP) without an eGFP expression construct (peGFPLuc), as shown in Panels A and B of FIG. 4, 2) 500 ng of the eGFP expression construct (peGFPLuc) without the eGFP-specific siRNA-expressing synthetic vector (H1-eGFP), as shown in Panels C and D of FIG. 4, 3) 500 ng the eGFP expression construct (peGFPLuc) with 200 ng of the eGFP-specific siRNA-expressing synthetic vector (H1-eGFP), as shown in Panels E and F of FIG. 4, or 4) 500 ng the eGFP expression construct (peGFPLuc) with 500 ng of the eGFP-specific siRNA-expressing synthetic vector (H1-eGFP), as shown in Panels G and H of FIG. 4. Flow cytometry was used to quantify the amount of fluorescence present at 96 hrs after transfection (FIG. 5).

As shown in FIG. 5, transfection of 293 cells with H1-eGFP alone resulted in a slight increase in fluorescence over mock-transfected cells. This increased fluorescence was not evident from fluorescent microscopic observation (FIG. 4B), and thus most likely represents non-specific autofluorescence. Transfection with the eGFP expression construct produced fluorescence in 46% of the cells assayed at 96 hrs post-transfection (FIG. 4, Panel D and FIG. 5). Co-transfection of the eGFP expression construct with either 200 ng or 500 ng of the H1-eGFP synthetic vector reduced fluorescence by 89% and 92%, respectively, as compared to cells transfected by the eGFP expression construct alone (FIG. 4, compare panels F and H with panel D; FIG. 5).

These studies demonstrate that PCR can be used to generate a compact vector comprising a linear dsDNA expression cassette, and that delivery of a vector of this type to cultured human cells can reduce the expression of a specific gene by over 90%. Thus, this compact vector represents a novel and efficient system for introducing dsRNA into cells.

### Example 3

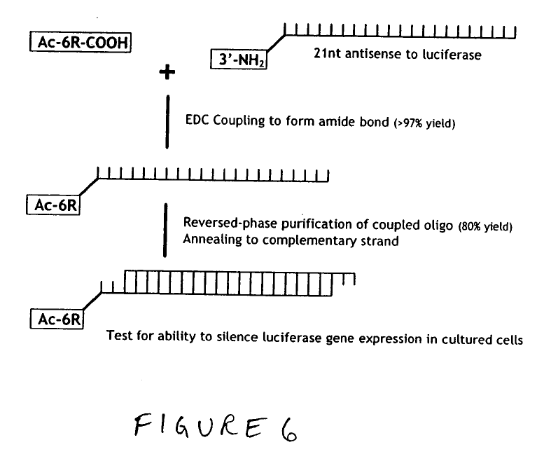

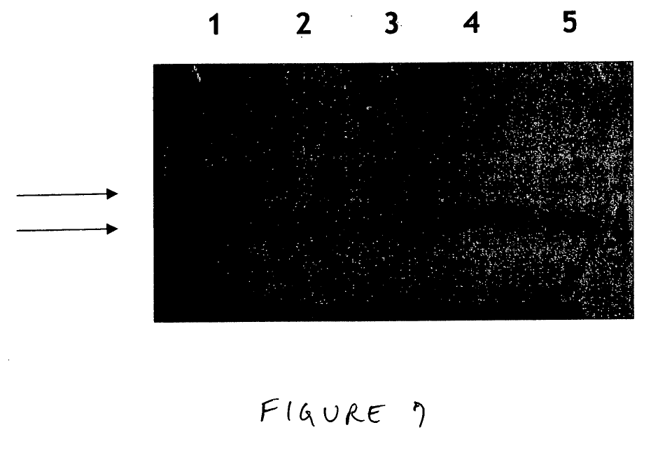

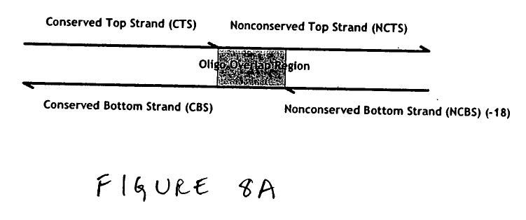

Coupling of Carboxy-containing Peptide Ligands to Amine-modifled Nucleic Acids Using EDC-mediated Coupling. To determine the efficiency with which peptide ligands might be coupled to nucleic acids, the synthetic PTD Ac-RRRRRR-COOH was coupled to a 21 bp antisense RNA molecule specific for the luciferase gene via an amino C3 group located on the 3′ end of the RNA molecule. This procedure is outlined in FIG. 6. EDC-mediated coupling of the peptide to the RNA molecule occurred at 97% efficiency. The coupled RNA oligonucleotide was then purified by reverse-phase chromatography (80% yield) and annealed to its complementary strand.

Coupling of the peptide ligand to the RNA molecule was confirmed by gel electrophoresis, as shown in FIG. 7. Uncoupled RNA is shown in Lane 1. RNA coupled to the Ac-6R PTD is shown in Lanes 2 and 4. RNA coupled to the 6CF-6R PTD is shown in Lanes 3 and 5. Coupling of the Ac-6R and 6CF-6R PTDs to a modified RNA was highly efficient (>97% efficiency).

### Example 4

A Compact Synthetic Vector May Be Created Without PCR By Multipart Strand Synthesis Using Multiple Overlapping Complementary Oligonucleotides. The upper limit of current oligonucleotide synthesis technology is approximately 135 bp. Thus, vectors exceeding this size limit may not be assembled merely by the annealing of two complementary strands. To overcome this limitation, PCR amplification was used to add the additional base pairs to the 95-mer primer oligonucleotides used to construct the compact vector depicted in FIG. 1. However, the use of PCR increases the potential cost of producing the vector, and also provides an opportunity for contamination of the vector by biological materials, such as nucleic acids, enzymes, lipids, or carbohydrates, which may hinder the rapid validation and approval of the vector for use in clinical settings.

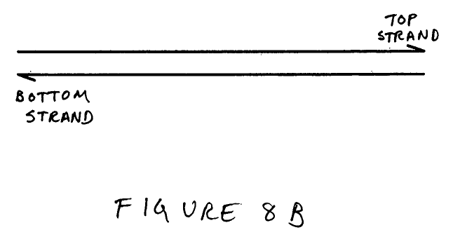

To avoid the need for PCR amplification, two alternative solutions, shown in FIG. 8, are proposed. In the first strategy, shown in FIG. 8A, the synthetic vector is assembled through the annealing of multiple oligonucleotides, each less than the approximately 135 bp limit of current oligonucleotide synthesis technology. The advantage of this strategy is that it requires no further engineering of the vector. For example, this strategy could be employed to generate vectors of the type depicted in FIG. 1 without the use of PCR. The multiple parts of each strand of the vector are held together by the Watson-Crick base pairing prior to entry into a cell, and the breaks in each strand at the junction of the multiple oligonucleotides can be repaired intracellularly by the cell's own DNA repair mechanisms provided that the oligonucleotides are 5′ phosphorylated.

The use of this multipart strand synthesis approach not only enables the generation of much larger constructs (>200 bp in size) using conventional DNA chemical synthesis machines, but may also be adapted for the rapid screening of various siRNAs and functional RNAs which can be generated from a synthetic cassette. For example, modular 5′ and 3′ segments of the cassette may be designed so that a wide variety of siRNA or functional RNA constructs could be tested merely by swapping various modules in the 5′ or 3′ portions of the cassette. This goal could be accomplished more cheaply and quickly than by synthesizing and purifying the whole transcribed and nontranscribed strands each time. Also, yields would be dramatically increased for the shorter oligonucleotides comprising the various modules.

### Example 5

A Compact Synthetic Vector May Be Created Without PCR By Whole Strand Synthesis Using Two Complementary Oligonucleotides. A second strategy that obviates the need for PCR amplification to generate the complete synthetic vector is shown in FIG. 8B. In this strategy, the synthetic vector is generated by annealing only two complementary synthetic oligonucleotides. As indicated above, because the current upper limit of chemical oligonucleotide synthesis technology is approximately 135 bp, further engineering of the vector to reduce its total length below approximately 135 bp is required to employ this second strategy. Examples of such synthetic vectors are described below.

The synthetic vectors created by chemical means are free of endotoxins, DNases, RNases, DNA, RNA, lipids, carbohydrates and proteins. Such vectors would also fail to engender the pro-inflammatory responses caused by the presence of non-methylated CpGs that are found in plasmids derived from prokaryotic sources. This lack of biological contamination greatly facilitates the validation of these vectors for clinical use after their efficacy has been demonstrated in animal models.

In the absence of the compact synthetic vector of the instant invention, the only methods for testing RNA silencing require either synthetic RNA duplexes/hairpins, which are expensive and unstable, or plasmid-based siRNA expression, which is time-consuming. Thus, the 50-70 nt delivery capacity of the compact synthetic vectors, which permits the delivery of a broad range of ssRNA hairpin/partial hairpin/non-hairpin transcripts for RNA interference or shorter functional RNAs, represents a cost-effective and efficient alternative to present methods for testing siRNA constructs. Various molecules could be rapidly screened in biological systems without the need for annealing, ligation, transformation, cloning, sequencing, expansion, purification and transfection, as is necessary for plasmid constructs to verify their ability to exert some biological effect through RNAi or some other means.

### Example 6

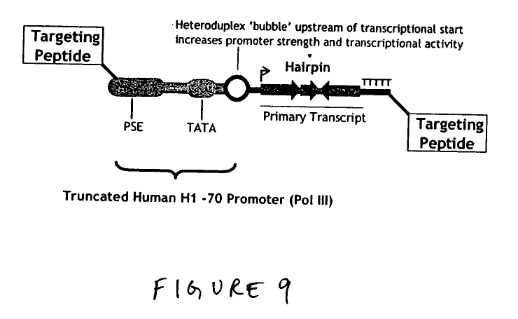

The Size of the Compact Synthetic Vector May Be Further Reduced by Alteration of the Human Pol III Promoter Region. Several portions of the vector depicted in FIG. 1 may be amenable to further alteration to reduce the overall length of the vector to below 135 bp. For example, the human H1 Pol III promoter employed in the vector may be truncated to delete the remaining internal control elements. As shown in FIG. 9, this approach has been used to remove approximately 30 bp from the approximately 100 bp human Pol III promoter. The net result of this deletion is a reduction in total length of the vector from approximately 160 bp to approximately 130 bp and in its molecular weight from approximately 110 kDa to less than 90 kDa. This reduction in total length minimizes overall net negative charge and may aid entry of the vector by ligand-independent and ligand-mediated pathways. Smaller vectors may also persist longer in vivo owing to their smaller size and lower anionic charge. Once internalized, these constructs also may enter the nucleus with greater efficiency than their larger counterparts. Nuclear targeting may be enhanced by coupling the dsDNA cassettes to ligands containing NLS-sequences.

The ability to construct the vector from completely synthetic oligonucleotides also permits the inclusion of both conventional oligonucleotide modifications at any position in the cassette as well as unconventional structures (e.g. hairpins, heteroduplex ‘bubbles’, 5′ or 3′ overhangs, etc.) that would be difficult if not impossible to achieve through biological means. For example, a heteroduplex “bubble” may be introduced upstream of the transcriptional start site to increase promoter strength and transcriptional activity. Such bubbles have been shown to increase promoter strength 100-fold in vitro. Also, fluorescent dyes, hairpins, phosphorothioate groups, non-natural bases, 5′ and 3′ overhangs, 5′ and 3′ modifications, and mismatches can be incorporated into either or both strands of the dsDNA. These modifications may improve the half-life of the constructs.

The compact synthetic vectors of the instant invention greatly facilitate the rapid validation of rationally-designed, synthetic promoters for driving expression of short RNAs. Such promoters may be derived from Pol III, Pol II or other suitable promoters. TATA boxes can be optimized for Pol II or Pol III expression and tissue-specific minimal promoter elements can be included, as well (i.e. myelin basic protein, metallothionein, NF1, PER1, etc.). Examples of suitable promoters are provided hereinbelow.

### Example 7

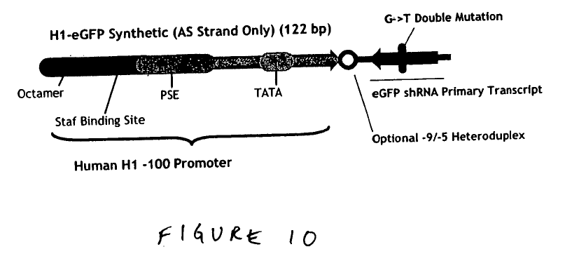

The Size of the Compact Synthetic Vector May Be Further Reduced by Alteration of the Portion of the Vector Encoding the Primary Transcript. In addition to engineering of the promoter region to reduce the overall length of the compact synthetic vector to within the confines of current oligonucleotide sequence technology, this goal may be achieved through alteration of the portion of the vector encoding the primary transcript. For example, as shown in FIG. 10, the length of the region from which the functional RNA molecule is transcribed my be reduced from approximately 50 bp to approximately 20-30 bp by modifying this region so that it contains only a partial RNA hairpin rather than the full hairpin as shown above in FIGS. 1 and 6. Because only the antisense strand is necessary to activate the RNAi pathway (Martinez et al., Cell 2002;110:563-74), the elimination of one strand from the RNA hairpin encoded by this region will permit reduction in the overall length of the vector by approximately 20-30 bp without affecting the vectors ability to elicit RNA interference. Alterations of the transcribed region may also be used to reduce the overall length of the vector when RNA molecules besides siRNAs are being transcribed. Thus, a similar approach may be used to reduce the overall size of vectors for the transcription of antisense molecules, ribozymes or other RNAs that retain function when reduced in total size to 20-30 bp. Obviously, both approaches, reduction in promoter size and reduction in the length of the transcribed region, may be employed either alone or in combination to achieve the desired reduction in overall vector length.

### Example 8

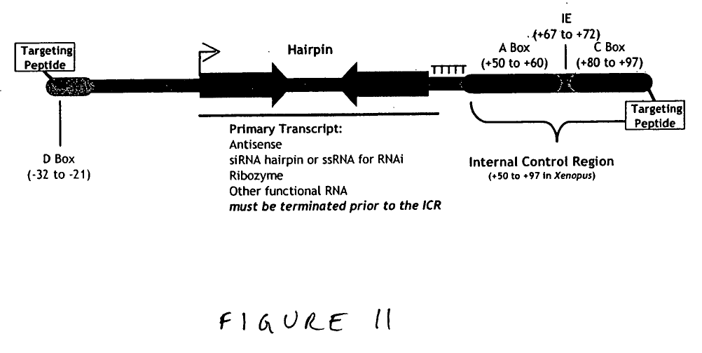

Compact Synthetic Vectors Containing the Type 1 Pol III Promoter. As shown in FIG. 11, the Type 1 Pol III promoter can be modified for the expression of functional RNAs, including RNAs that mediate RNAi. To maintain a compact structure, non-conserved, internal elements can be removed and substituted with the functional RNA transcript of interest, between position +1 to the beginning of the internal control region at position approximately +50. This spacing permits the insertion of a single stranded RNA for RNAi or antisense, an siRNA hairpin, or a ribozyme.

Transcription is accurately initiated from the promoter at a fixed distance from the internal control region (ICR). Transcription is terminated by introducing a run of 4 to 6 consecutive thymidines at the end of template. The internal control region, which comprises a series of three highly conserved elements (‘A Box’, ‘Intermediate Element (IE)’ and ‘C Box’) spanning from +50 to +97 in Xenopus must be retained for proper recruitment of the basal RNA polymerase III apparatus. An additional element, the ‘D Box’, is a conserved motif in other eukaryotes, including humans, that is required for adequate expression in this system. Extraneous sequences 5′ to the D-Box and 3′ to the ICR will be eliminated, except for the nucleotides required for optimal expression from the construct, if necessary. The length of this cassette is 129 bp, which is below the current 135 base limit obtainable by chemical DNA synthesis methods. Due to the fact that this cassette is chemically synthesized, it may be modified in any of the ways described above, including the addition of targeting peptides.

### Example 9

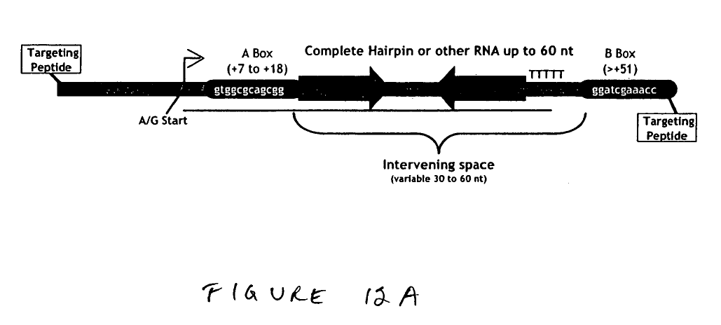

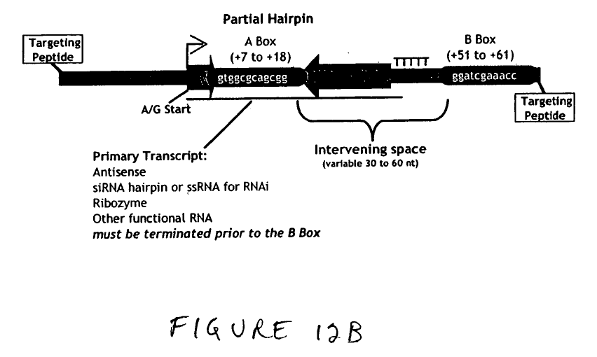

Compact Synthetic Vectors Containing the Type 2 Pol III Promoter. The tRNA promoter has been widely used for expression of functional RNAs, including antisense and ribozymes placed downstream of the intact or slightly modified tRNA. As shown in FIG. 12, this promoter may be internally modified cassette for expression of functional RNA, including RNAs that mediate RNAi. tRNAs are typically 72-73 nt in their mature form. To maintain a compact structure, non-conserved, internal elements will be removed and substituted with the functional RNA transcript of interest, between approximately positions +1 to +6 and +19 to +50, based on conserved elements present in the human tRNA^{met} expression cassette. In the case of tRNAs, two highly conserved elements, the ‘A Box’ (GTGGCGCAGCGG; SEQ ID NO:5) and the ‘B Box’ (GGATCGAAACC; SEQ ID NO:6) will be retained in order to properly recruit the Pol III transcriptional apparatus. Variations on this spacing can take into account the variability present in various eukaryotic tRNAs. For example the distance between the A Box and B Box may vary from approximately 30 to 60 bp, owing to the presence of a splice site.

In the example depicted in FIG. 12A, a complete hairpin can be inserted downstream of the A Box that terminates prior to the B Box, Alternatively, as shown in FIG. 12B, a partial hairpin can be inserted in which the first 6 bp of the mature transcript basepairs with a 19-21 nt antisense strand, the A Box actually serving as the stem loop structure in this example. In both cases, the primary transcript would be processed by RNase III into a mature dsRNA that can mediate RNAi. As for the type 3 Pol III promoters, transcription is accurately initiated from the promoter at a fixed distance from the A Box. Transcription is terminated by introducing a run of 4 to 6 consecutive thymidines at the end of template. Extraneous sequences 5′ and 3′ to the conserved regions should be eliminated, save for nucleotides required for proper RNA expression. Although specific sequence elements upstream of the start site are not defined, some 20 to 50 bp are likely to be required for the Pol III apparatus to dock to the expression cassette.

The total length of this synthetic cassette would range from approximately 90 to 130 bp, which is below the current 135 base limit obtainable by chemical DNA synthesis methods. Due to the fact that this cassette is chemically synthesized, it also may be modified in any of the ways described above, including the addition of targeting peptides.

### Example 10

Compact Synthetic Vectors Containing a Pol 11 Promoter. The RNA polymerase II promoter is the most widely used promoter system for gene expression. Particularly strong promoters, such as those that are virally derived (e.g. CMV, SV40, Moloney leukemia virus, etc.), yield excellent expression levels in a variety of cell types in vitro and in vivo. These promoters generally run several hundred base pairs in length (590 bp in the case of the CMV immediate early promoter). There has been little incentive to reduce the size of the promoters used in vitro and in vivo, as these lengths are relatively small in relation to the typically used plasmid sizes (2-10 kilobases). However, in the case of a linear, synthetic dsDNA expression cassette, the most compact, yet powerful promoter is desirable to drive high levels of downstream expression.

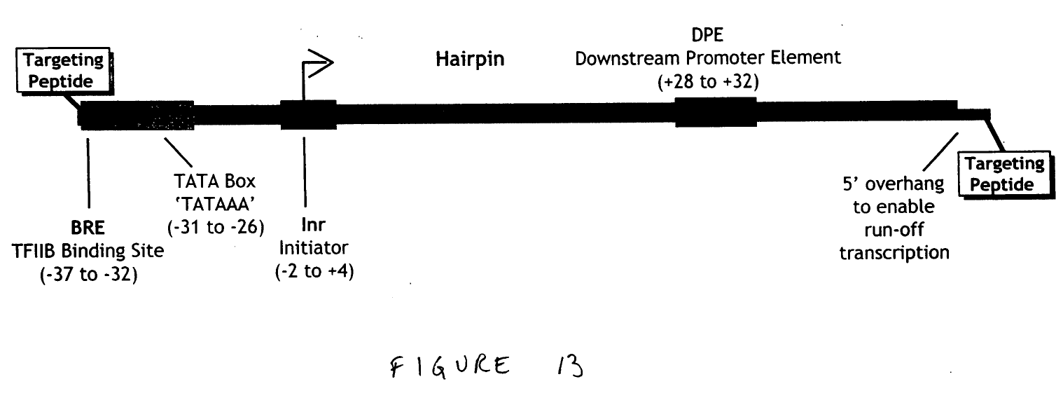

To achieve this goal, consideration of the inclusion only of those core Pol II promoter elements required for adequate expression (BRE, TATA, Inr, DPE) may be critical. Since promoters can assume many forms, with some containing none of the core elements, and other containing only some elements (e.g. BRE and TATA box only), various constructs will need to be tested. One such construct is shown schematically in FIG. 13. For the purposes of expressing RNA for RNAi, it is important to recognize that nearly all Pol II constructs are 5′ capped, which would block incorporation into the RISC complex if the antisense strand happens to be capped. Also, to enable efficient run-off transcription on a linear template, a 5′ overhang at the end of the dsDNA cassette may be necessary (Izban et al., J. Biol. Chem. 1995;270:2290-2297). This enables the precise and accurate termination of the transcript, without need for the typical polyadenylation signal found on most Pol II mRNAs. Additional enhancer and non-conserved sequences may be necessary upstream of these core promoters in order to facilitate binding of basal Pol II factors. In total, the promoter and transcript must be contained within approximately 135 bp in order to be synthesized as a single strand, or under ˜260 bp if four overlapping annealed oligonucleotides are to be used.

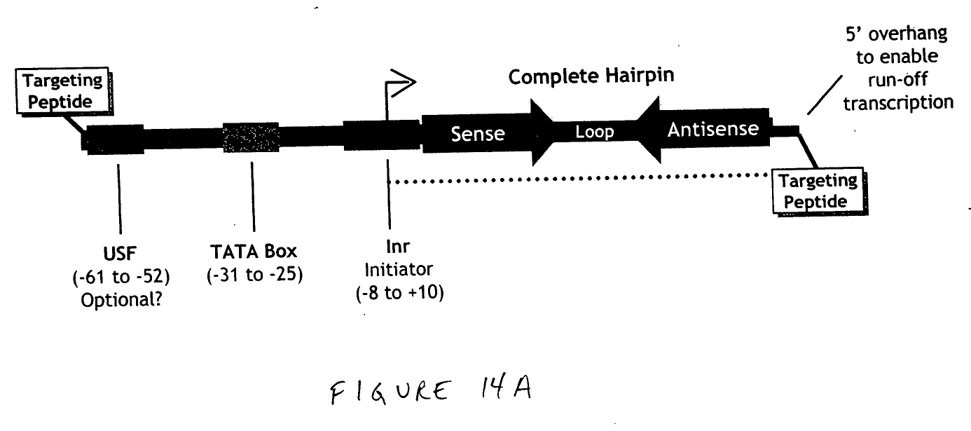

One example of a powerful yet compact promoter is the adenovirus-2 major late promoter (AdML2), which nominally consists of a TATA Box with flanking sequences (−38 to −10), an upstream stimulatory transcription factor binding site (USF; −61 to −52) and an initiator element (−8 to +10) for expressing downstream siRNA hairpins and partial hairpins (Wang et al., Biochimica et Biophysica Acta 1998;1397:141-145). Two variants of a synthetic vector containing a polymerase II core promoter based on AdML2 are shown in FIG. 14. Note that in vectors of this design, a single antisense strand for mediating RNAi is not possible due to the 5′ capping of Pol II transcripts, which would block its incorporation into the RISC complex. However, 5′ modification of the sense strand of a hairpin or partial hairpin should not interfere with the antisense strand incorporation into RISC, which should be cleaved by RNase III into a RISC-competent ssRNA form (Martinez et al., Cell 2002;110:563-574; Zeng et al., Mol. Cell 2002;9:1327-1333). In the case of the AdML2 promoter, the primary transcript will unavoidably contain part of the initiator sequence fused to the sense strand of the partial or complete hairpin. This should not present a problem, following RNase III processing, as the correct antisense strand should be liberated for mediating RNAi.

Inclusion of cell-type specific elements in the promoter may be useful to limit expression to only target organs and tissues. Examples of notable tissue-specific minimal promoter elements include, but are not limited to, those found in the genes encoding prepro-endothelin-1, myelin basic protein, metallothionein, the neurofibromatosis-1 (NF1) protein, growth hormone factor 1 (GHF-1), peripherin, fibroin, JC virus (JCV) proteins, and the period-1 (PER1) protein. All of these minimal promoters are compact and may be sufficient for Pol II tissue/organ-specific gene expression.

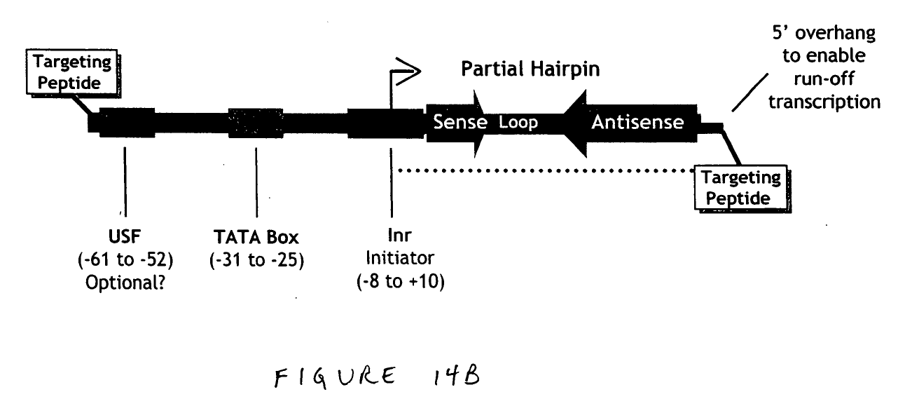

One example of how these minimal promoters might be employed for the expression of functional RNAs in the context of a compact synthetic vector is shown in FIG. 15. This example incorporates the human papovavirus JC core promoter (JCV; TTTTTTTATATATACAGGAGGCCGAGGC;SEQ ID NO:7). The JCV promoter is glial-specific in its expression pattern and exceptionally compact. It contains only a 7/8-bp poly(T) region followed by a TATA box and confers glial-specific gene expression patterns of downstream reporters (Krebs et al., J. Virol. 1995;69:2434-2442). In total, the entire control region spans only 28 bp and initiates at multiple positions (+5, +25, and +40 bp) from the center of the TATA box. Due to the presence of multiple start sites in vivo, it is conceivable that an extraordinarily compact linear dsDNA cassette of <60 bp could be constructed when using a partial hairpin if the +5 initiation site from the TATA box functions in this context.

As in the case of Pol III promoters, any kind of conceivable oligonucleotide modification can be incorporated in these compact Pol II promoters, due to the chemical synthesis approach for these cassettes. These include heteroduplex bubbles, overhangs, unnatural bases and linkages, etc. The inclusion of a heteroduplex should increase expression levels by reducing one of the key rate-limiting steps for transcriptional initiation—the melting of the promoter DNA by the Pol II machinery. Again, as with compact synthetic vectors containing Pol II promoters, there is no real limit to the type of RNA cargo to be expressed, whether it is an antisense RNA, a full or partial hairpin for RNAi, a modified microRNA, a ribozyme, etc., provided that the total size of the vector lies within the current capabilities of oligonucleotide synthesis. The primary advantage Pol II promoters provide over those driven by Pol III is the potential for tissue-specific control of expression.

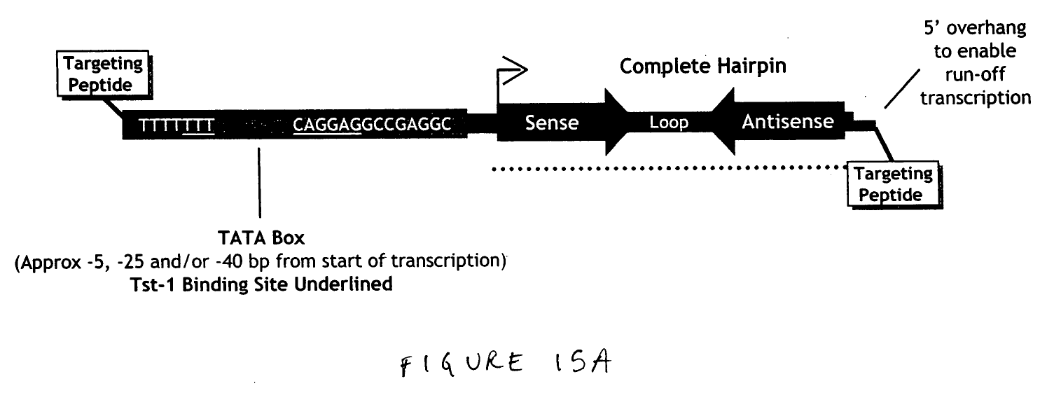

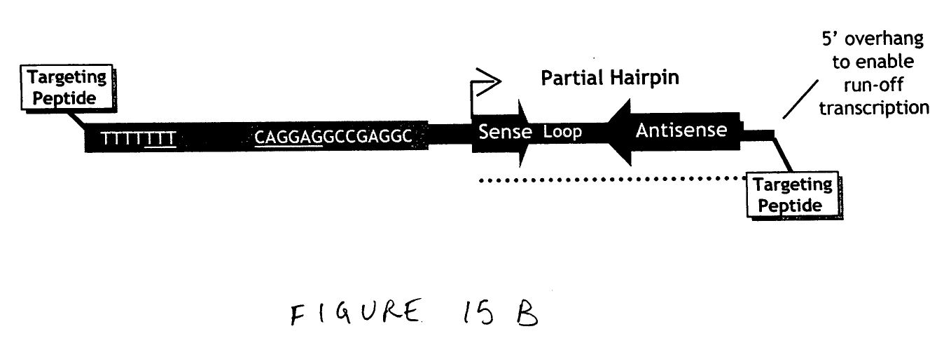

One of the limiting factors for Pol II promoters is their ability to recruit the Pol II apparatus to the DNA in order to initiate transcription. Inside the cell, a dsDNA must effectively compete with endogenous genes for the Pol II transcriptional machinery. One way in which this may be overcome is through the use of a covalently tethered artificial transcription factor, such as the acidic domain from VPI6. Apparent from the paper by Stanojevic and Young (Biochemistry 2002;41:7209-7216), it may be possible to tether directly to a short dsDNA synthetic expression cassette a transactivator peptide (CGSDALDDFDLDML) to an amine-modified oligo by EMCS (succinimidyl-6-maleimidlyhexanoate; Molecular Probes) by a cysteine to amine-bridge. Other covalent linkages may also be possible. One such vector is shown in FIG. 16. The peptide sequence for the AD-16 polypeptide is CGSDALDDFDLDMLGS (SEQ ID NO:8). The peptide sequence for the AD-29 polypeptide is CGSDALDDFDLDMLGSDALDDFDLDMLGS (SEQ ID NO:9). The direct recruitment of a strong transactivator to a TATA box in a Pol II context may enable the expression of a silencing RNA or other functional RNA with only minimal upstream sequences from the TATA box required.

To validate this concept, a synthetic vector may be generated in which a minimal Pol II promoter (a basal adenovirus E4 promoter, a minimal TK promoter, etc.) drives expression of a DsRed-Express reporter gene with SV40 Early polyA sites and terminating downstream of the 3′ end of the full-length RNA (total length ˜1 kb). The construct would be made by annealing modified oligos into the upstream MCS of the promoterless Clontech vector, pDsRed-Express-1 and performing PCR. Because the vector has only a minimal promoter, for a crude assay, separation of the template plasmid from the PCR product will not be necessary (no gel excision required for the initial experiments). The linear double-stranded product would be generated by PCR using a modified forward oligo with a C12 amino extension for coupling to the VP16 transactivator peptide. The forward oligo may even be modified exactly as described in Stanojevic and Young (Biochemistry 2002;41:7209-7219) as a control that would mimic their structure and design as closely as possible. The reverse oligo would contain a FITC or other fluorescent green/far red tag to monitor internalization of the double-stranded product. This coupling would be done via sulfo-EMCS and the transfected product would be checked for red fluorescence 8-12 hours post-transfection (maximal expression at 24-30 hours) and compared to an uncoupled control.

If the construct is functional, then two overlapping synthetic oligos with the sense strand modified with a 5′ amino C12 and +/− a heteroduplex bubble upstream of the start site can be annealed to an antisense strand with a 5′ overhang for run-off transcription and +/− a C73′ amino modifier. The antisense oligo may even have a 5° Cy3 label so that its internalization can be monitored. In this manner, both strands may be coupled to the VP16 transactivator peptide, if desired. The inclusion of the heteroduplex may facilitate enhanced transcription off the synthetic Pol II promoter. A downstream full or partial hairpin RNA (dsDNA length ˜95) for RNAi can be generated against the eGFP reporter gene (pEGFPLuc reporter plasmid co-transfected) as proof that it is functional. This short construct might be able to enter cells in culture without a transduction/targeting domain, but also consider appending a PTD/RGD/NLS domain to facilitate entry into cells.

For increased stability, the D-form of the VP16 transactivator domain peptides can be constructed, as previously described (Nyanguile et al., Proc. Natl. Acad. Sci. USA 1997;94:13402-13406). Also mentioned by Nyanguile and colleagues is the lack of effect of the L-form of the peptide, but not the D-form, suggesting that intracellular proteolysis may play a major role in controlling the results observed (i.e. lack of observable effect; proteasome inhibitors can be tested as a control). The final constructs can be labeled with Alexa Fluor 488 using the ULYSIS Nucleic Acid Labeling Kit (Molecular Probes) to monitor uptake, if desired. This example of a synthetic Pol 11-driven dsDNA expression cassette would be constitutively active and not tissue-specific.

### Example 11

Compact Synthetic Vectors Containing Inducible Pol II or Pol III Promoters. For an inducible gene expression system, a binding factor that interacts with upstream elements 5′ to the start of transcription may be employed (Ohkawa and Taira, Human Gene Ther. 2000;11:577-585). Factors that bind DNA in the absence or presence of a ligand, such as dexamethasone (glucocorticoid receptor), doxycycline (Tet system), 17β-estradiol (estrogen receptor), ecdysone, etc. can control gene expression by sterically blocking Pol III assembly in the region 5′ to the transcriptional start. It is known that even in promoters that are downstream of the start site, the Pol III apparatus must assemble some 30-40 bases upstream. This can work for both induction or repression in the presence of a ligand; the system can function in both ways, as shown by the tet on/off and tet off/on designs. For a tet-based system, the introduction of the artificial repressor/activator is also required, however. These approaches should also work for controlling gene expression in Pol II systems.

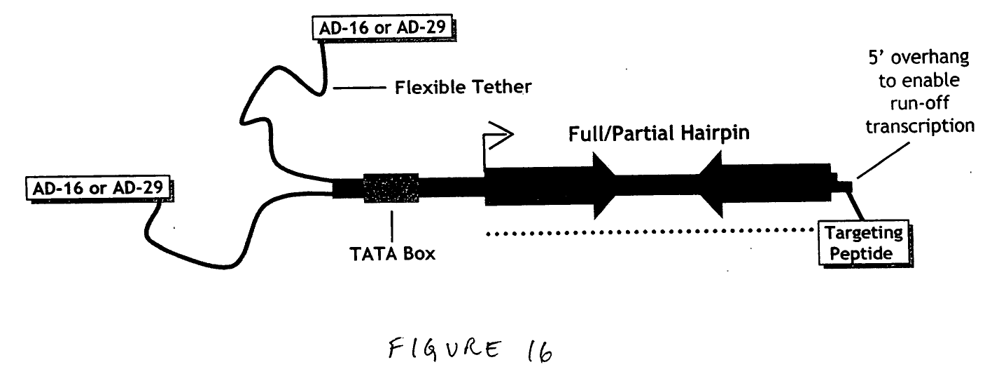

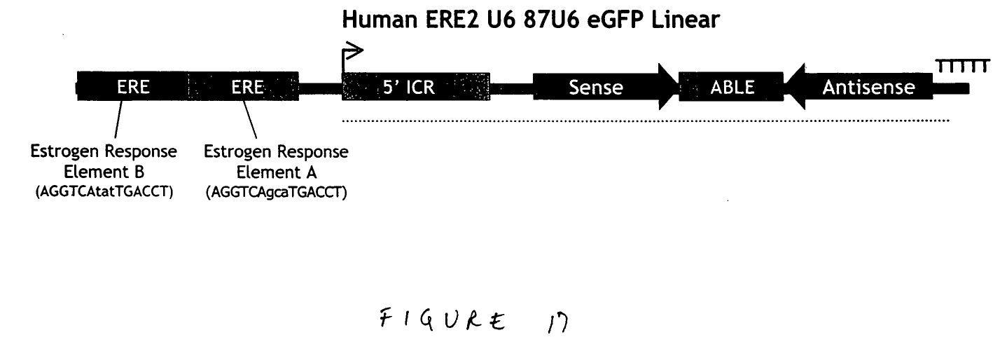

A hormone receptor-based system also may be able to control expression with specific unmodified cell lines or cell lines engineered to express the hormone receptor. Estrogen-mediated repression of gene expression by addition of 17β-estradiol may be possible, and a system controlled by estrogen is depicted schematically in FIG. 17 as an example of an inducible system. In this figure, the nucleotide sequence of Estrogen Response Element A is AGGTCAGCATGACCT (SEQ ID NO:10), while the nucleotide sequence of Estrogen Response Element B is AGGTCATATTGACCT (SEQ ID NO:11). The functioning of this system in the presence and absence of 17β-estradiol is shown in FIG. 18. When choosing the appropriate receptor/ligand pair for modulating gene expression, it is important to choose those drugs that will have minimal side effects and rely on the lowest ligand concentrations possible. Tight control over the time course of induction of gene expression, easy delivery of the ligands, low ligand costs, and low leakiness of gene expression must be also considered. Also critical are tissue-specific distributions of the relevant steroid hormone receptors if endogenous cellular receptors are to be used, otherwise the synthetic receptors must also be expressed in the target cell (e.g. tet repressor). These constructs are useful in vitro for controlling temporal silencing events of targeted genes. However, they may be also useful in gene transfer and developmental biology studies. For a review, see Lewandowski, Nat. Rev. Genet. 2001;2:743-755.

### Example 12

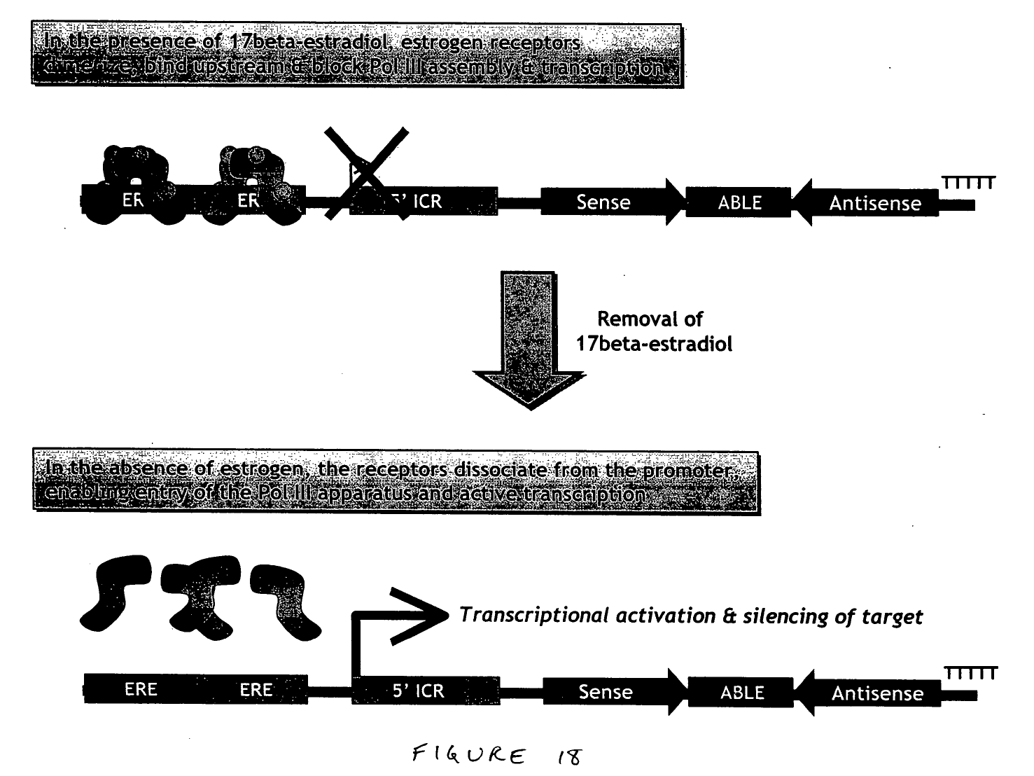

Compact Synthetic Vectors Containing a Variant Type 3 Pol III Promoter. A human U6 variant of the Type 3 Pol III Promoter (87U6) that has only an internal promoter (Tichelaar et al., Biochemistry 1998;37:12943-12951), and thus shares several characteristics of type II Pol III promoters, may be useful for a synthetic compact may be useful for a synthetic vector. A diagram of the expression system is shown in FIG. 19. As in other gene-internal Pol III promoters, transcription is initiated at a fixed distance from conserved elements. In this case, a 5′ Internal Control Region (ICR; GTGCTTGCTTTGGTAGCACA; SEQ ID NO:12) and a downstream ABLE box (AAGATTAGCACAGT; SEQ ID NO:13) are required for active transcription and accurate initiation. The space intervening the 5′ ICR and the ABLE sequences provides sufficient space to place a sense strand of a hairpin, and the antisense strand follows the ABLE box.

In this construct, the ABLE box serves as a hairpin loop for the RNA transcript, which is used for RNAi. Transcription is precisely terminated by a run of thymidines. Notably, since conserved 5′ gene-external elements are not required, the flanking sequence may simply be comprised of the REPori sequence if used in a micro-circle expression context. Hopefully, this additional upstream DNA will provide the surface for docking of Pol III elements and still enable episomal semi-conservative replication in vivo without adding too much additional sequence. This synthetic vector may be more compact than variations based upon other Pol III systems, while providing higher expression levels than the Type 1 and Type 2 Pol III promoters.

### Example 13

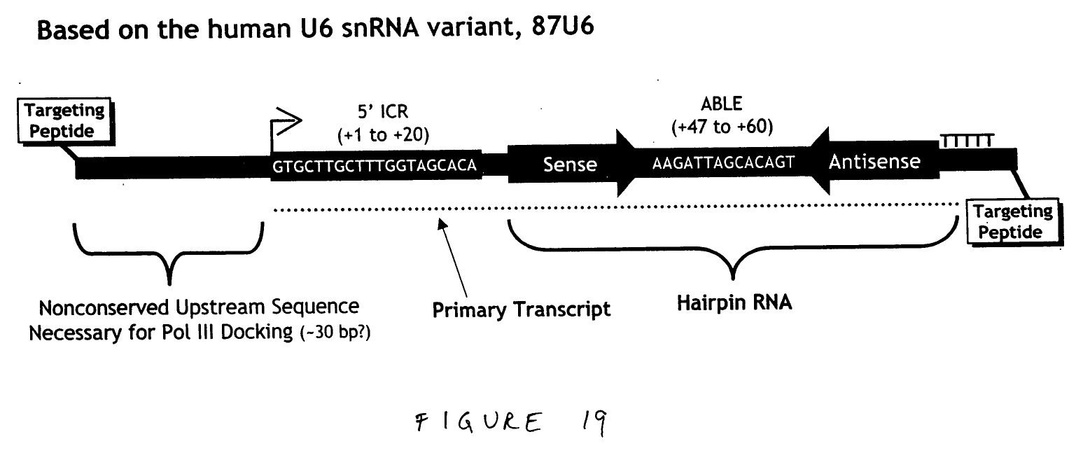

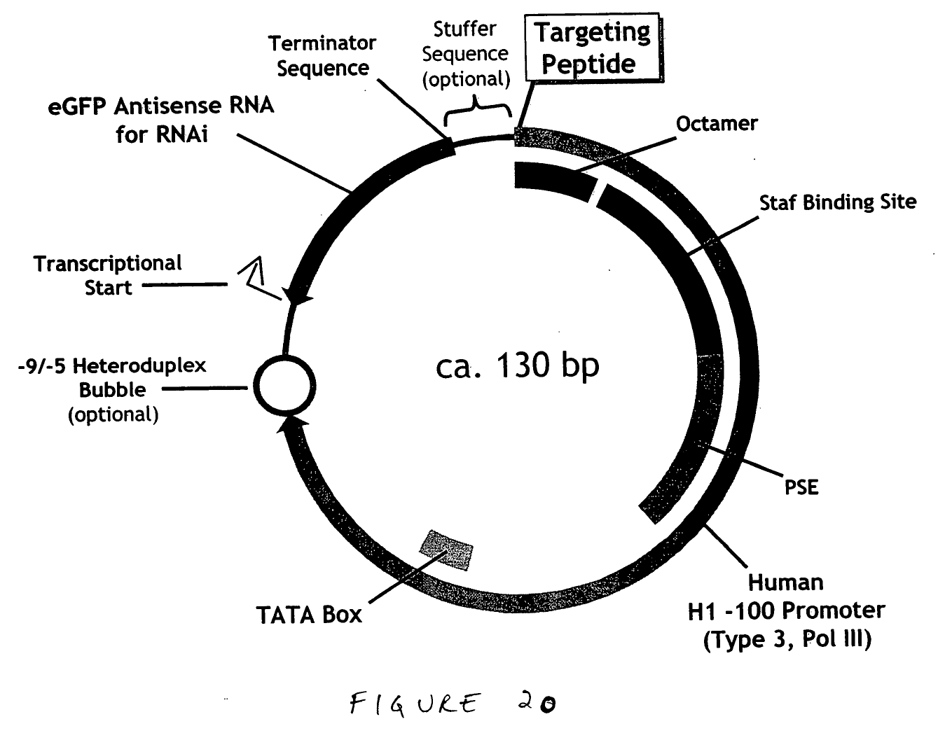

Compact Synthetic Vectors May Be Linear or Circular. The examples of compact synthetic vectors discussed hereinabove have generally been depicted as linear, double-stranded molecules. However, compact synthetic vectors of the instant invention may, in fact, exhibit greater stability and/or higher expression when circularized, because the strain generated by circularization may facilitate promoter melting. Thus, linear dsDNA expression cassettes may be circularized as single units, or concatemerized (homoconcatemerization using the same repeat unit, or heteroconcatemerization using dsDNA cassettes that silence different genes). Naturally, this approach will limit modification of the 5′ and 3′ ends of the oligos; attachment of targeting peptides will have to be performed on internally-modified bases, or by modified oligonucleotides that bind to unpaired, heteroduplex regions. Both of these solutions are readily available. FIG. 20 shows a schematic representation of the circular form of a compact synthetic vector comprising the H1 Pol III promoter and encoding an eGFP antisense molecule for RNAi.

DNA ligase treatment may be used to achieve circularization of sticky or blunt-ended constructs with appropriate 5′ phosphorylations. The use of ligase can open up the ability to generate larger circular expression cassettes that are made up of multiple oligos. Despite their circularization, these ‘mini-plasmids’ or ‘micro-circles’ are preferably far smaller than previously described plasmids for mediating RNAi, and are approximately 1-2 logs smaller than conventional plasmids, with the inherent potential for greater uptake into cells in vitro and in vivo.

This approach may be particularly amenable to Pol III promoters as they terminate precisely at a run of thymidines. Additional ‘stuffer’ DNA sequence between the end of the primary transcript and the beginning of the promoter may be required to reduce steric interference between the bound Pol III factors and the transcribing RNA polymerase and to reduce torsion generated by DNA unwinding from the transcriptional machinery. Pol II promoters may require a more elaborate termination system, with sequences generally too large to include in the synthetic vectors of the instant invention, even when a compact polyadenylation signal is included. See Xia et al., Nature Biotech. 2002;20:1006-10. Nevertheless, it still may be possible to use a Pol II system in synthetic micro-circles (e.g. using the microRNA system, which is rather compact).

A 36 bp mammalian origin of replication (REPori A3/4) has recently been identified and the sequence has been published (Matheos et al., Biochim. Biophys. Acta. 2002;1578:59-72). This ori acts by recruiting the Ku proteins and also Oct1, as part of a complex, and appears to function efficiently in larger plasmids and YACS in vitro. This small ori, which is available from REPLICor (www.replicor.com), can be readily incorporated into circular, compact synthetic vectors, thereby enabling long-term propagation of the construct into daughter cells in vitro and in vivo by semi-conservative replication. Using this ori, circular dsDNAs are maintained outside the genome in an episomal form which is not integrated.

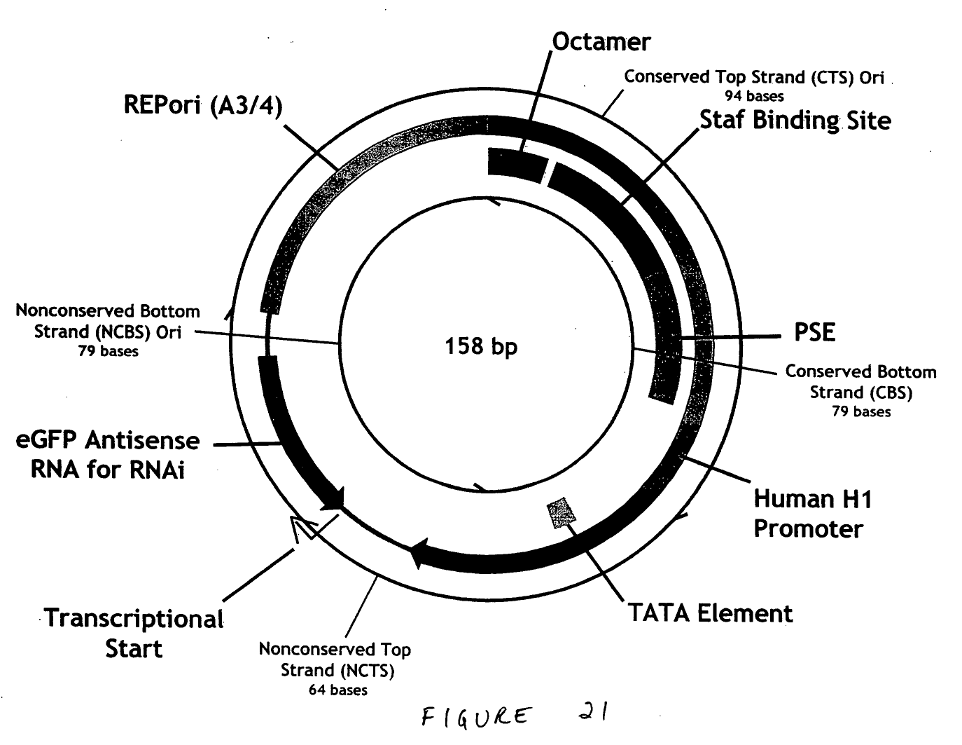

An example of a micro-circular form of the compact synthetic vector containing a replication origin is shown schematically in FIG. 21. Placed downstream of the RNA expression cassette and upstream of the promoter, the presence of the ori may facilitate enhanced gene expression of the synthetic cassette without selection. Due to its recruitment of numerous protein factors in trans, it may require additional spatial separation from the promoter and the RNA expression cassette. Thus, additional stuffer DNA may be necessary in order to provide the ori with sufficient room to confer its replication functions while not interfering with activity of the promoter and synthesis of the RNA transcript. At present, the cell- and species-specificity of the ori is not fully characterized. Moreover, this ori does not act as a centromere and does not have telomeric functions. Thus, in the absence of selection, segregation occurs efficiently, but not absolutely to all daughter cells (90% efficient).

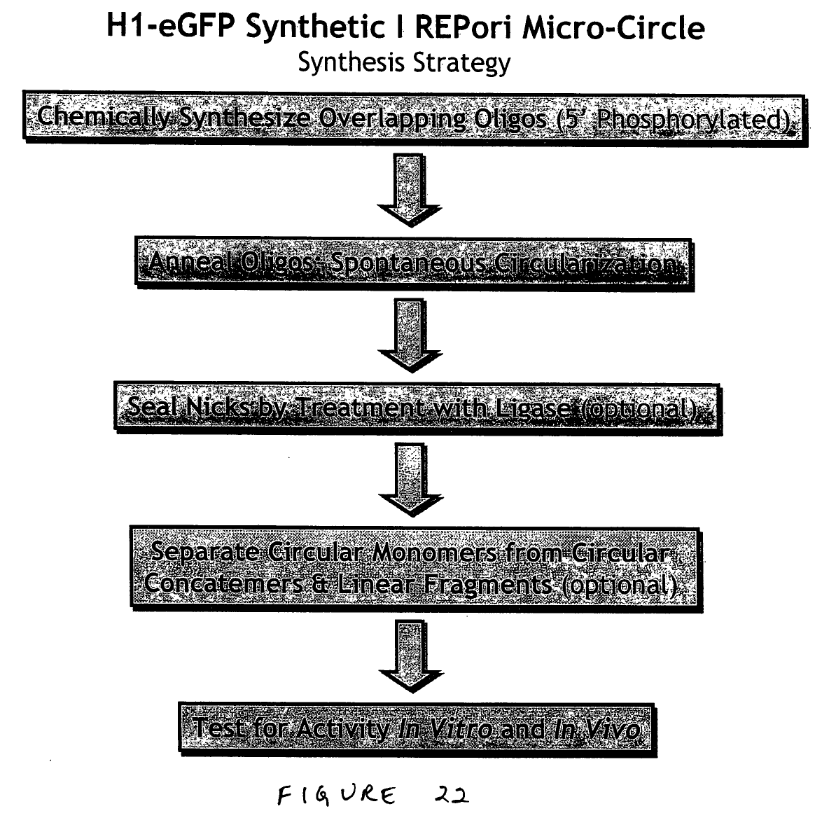

A strategy for the synthesis of ori-containing microcircular forms of the compact synthetic vector is shown in FIG. 22. Given a suitably overlapping series of oligonucleotides, spontaneous self-ligation and/or concatemerization may occur that is sufficient to generate nicked-open circle dsDNAs that are still functional. Once inside a cell, they may either function as nicked constructs, depending on the location of the nicks, or simply be repaired by the host cell DNA repair machinery, as described above for linear versions of the compact synthetic vector. If ligases are used, the concern over the specific location of nicks is obviated. Also, multipart assembly in a circular construct can conceivably contain significantly more than 2 or 4 oligos; as many overlapping oligos as needed can be employed, enabling the generation of relatively large dsDNA micro-circles (>135 bp and generally less than 1 kb in size)—with or without the use of ligase. At the same time, avoidance of use of PCR will enable substantially greater yields of circular dsDNA that can be used without a bacterial or eukaryotic expression system to amplify the copies. Importantly, the abrogation of the need to use a living system to amplify the construct eliminates the need for large origins of replication as well as selective markers and their associated promoters which have no function once in their target mammalian systems. These elements alone commonly occupy greater than 1 kb of a plasmid.

Additionally, all the other advantages of a synthetic approach that apply for the linear dsDNA apply here as well, including the possibility of incorporating modified residues that can enable the coupling to targeting ligands (PTDs, RGD, folate, etc.) and/or peptides with NLS to enhance nuclear import or transactivator functions. However, the presence of non-natural DNA modifications may block or impair DNA replication; such modifications will have to be chosen judiciously. Tradeoffs can obviously be made between the requirement of the vector to replicate and the need to target or possess other synthetic functions not present in naturally occurring circular dsDNAs. Careful placement of synthetic modifications may enable efficient targeting to a cell and appropriate replication once inside a cell, with the modified bases replaced by normal residues by the DNA repair machinery.

Initially, the activity of a micro-circular expression system derived from PCR fragments which have compatible cut sites at their 5′ and 3′ ends will be tested. These purified dsDNA fragments can be cut with the appropriate enzymes to generate sticky ends, and then annealed. Alternatively, phosphorylated oligos used in the PCR can be used to blunt ligate the ends without need for restriction digestion. The microcircle may initially contain a CMV-eGFP-Poly A expression cassette and the REPori ligated to form a circular construct. Its persistence in cell culture will be compared to the linear PCR product, the parental plasmid, and a circularized micro-circle without the REPori (or with an irrelevant sequence). Confirmation of replication of the episomal micro-circle can be confirmed, as described by Matheos et al. (Biochim. Biophys. Acta. 2002;1578:59-72).

### Example 14

Compact Synthetic Vectors Can Be Used To Deliver an RNAi That Silences Expression of the Human β-catenin Gene. Human β-catenin 1 has been chosen as one example of a target gene for RNA silencing by use of the compact synthetic vector. Mutations of this gene, which is an adherens junction protein, as well as of associated genes in the signaling pathway, are associated with colorectal cancers, prostate cancers, hepatoblastomas, hepatocellular carcinomas, ovarian carcinomas, and pilomatricomas. In adenomatous polyposis of the colon, the APC gene is mutated and unable to downregulate β-catenin signaling. In colon carcinomas lacking APC, constitutively active β-catenin/Tcf4 complexes are found. Restoration of functional APC downregulated β-catenin, suggesting that constitutive expression of Tcf4 gene products via dysregulated β-catenin is one of the early steps in carcinogenesis of the colonic epithelium. Likewise, activating mutations in β-catenin also lead to colorectal tumors, despite the presence of intact APC function. Furthermore, constitutive activation of the β-catenin signaling pathway also blocks the ability of stem-cells to differentiate into all three germ layers. Thus, modulating this pathway through RNAi presents an attractive target for cancer therapies, particularly of the colon, as well as controlling embryonic stem-cell differentiation.

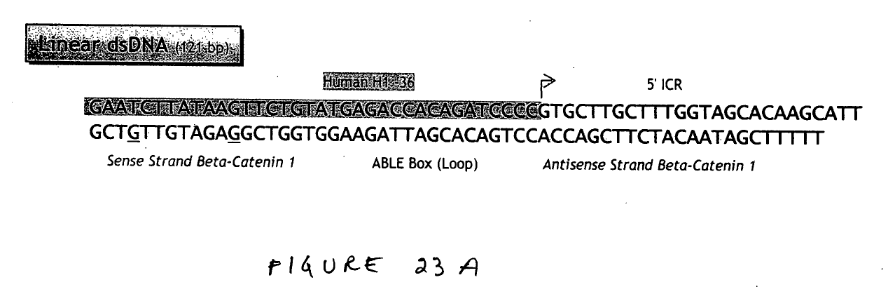

FIG. 23 depicts one strategy for the generation of a hairpin RNAi for the silencing of the β-catenin 1 gene. The presence of hairpin mismatches increases oligonucleotide synthesis yields and reduces cruciform DNA formation during hybridization. Changes made on the sense RNA strand do not adversely affect RNAi. Also, it has been shown that a 6-base 5′ overhang on a silencing RNA hairpin does not block silencing activity (Jacque et al., Nature 2002;418:435-438). Thus, the primary RNA transcript shown in FIG. 23 will likely serve as a substrate for RNase III, yielding the mature silencing transcript shown in the bottom panel of this figure.

### Example 15

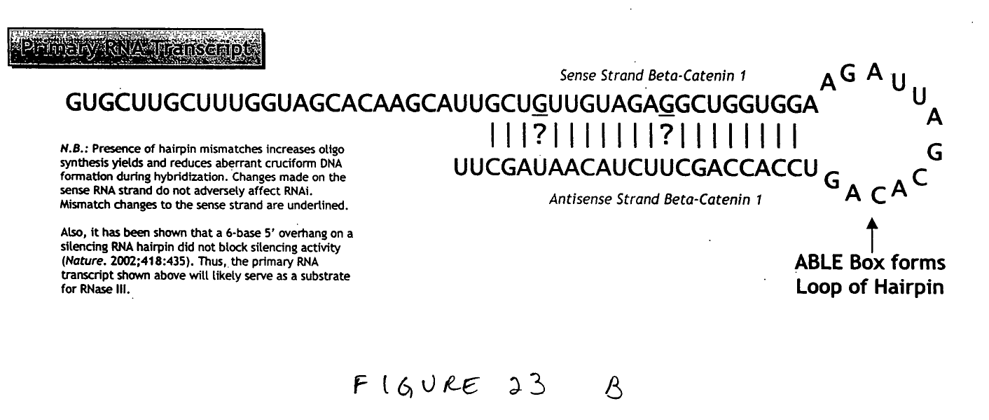

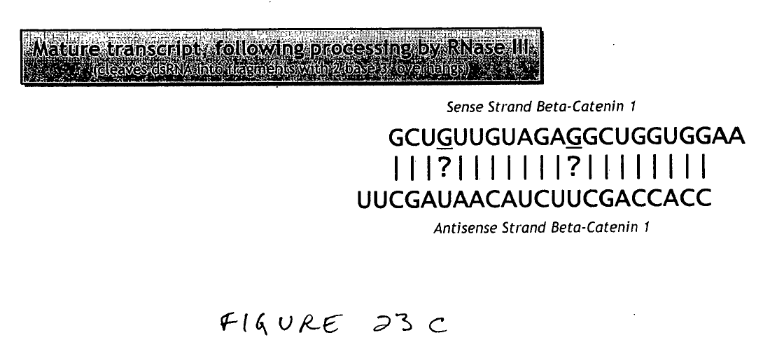

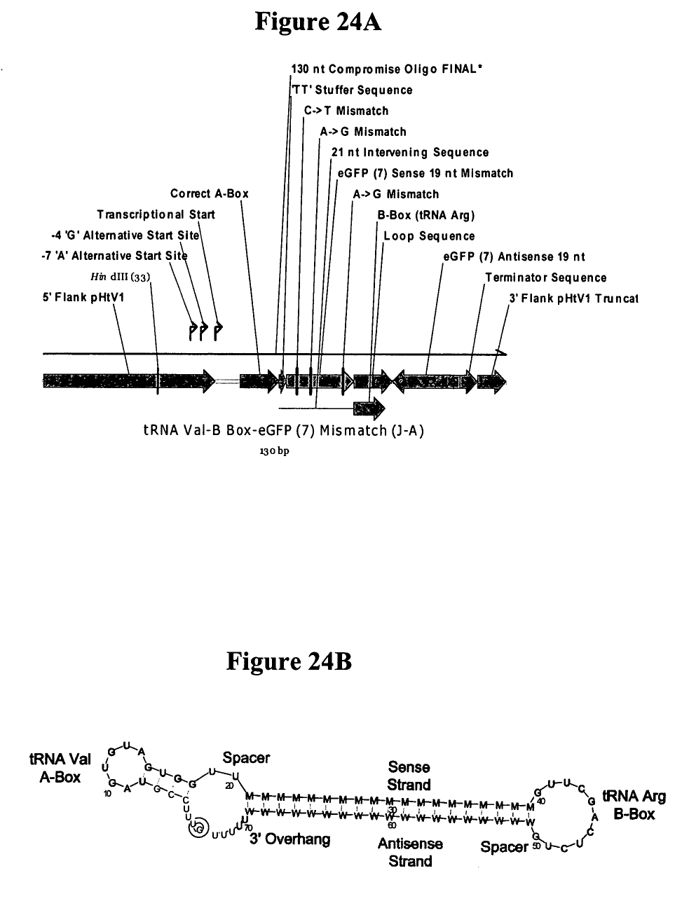

Silencing of Expression of the Green Fluorescent Protein (GFP) Gene in Cultured Human Cells by Transfection of Compact Synthetic Vectors That Express a siRNA Specific for the GFP Gene. Additional in vitro studies were performed to examine the functionality of other RNAi expression cassettes (RECs). The first of these, labelled as “tRNA Val-B Box-eGFP (7) Mismatch (J-A)” in FIG. 24A, is based on the human tRNA valine internal promoter (Genbank Acc. HtV1). The 130 bp sequence (5′-TTCAGGACTAGTCTTTTAGGTCAAAAAGAAGAAGCTTTGTAACCGTTGGTTTCCGTA GTGTAGTGGTTGAATGGCGTCAAGGTGGACGTTCGACTCTGGTTCACCTTGATGCCG TTCTTTTTCTATCGCT-3′; SEQ ID NO:35) contains a 5′ hairpin comprising the native tRNA Val “A-Box” (5′-TAGTGTAGTGG-3′; SEQ ID NO:36), and a loop sequence comprising the “B-Box” from human tRNA Arg (5′-GTTCGACTC-3′; SEQ ID NO:37). The sequence contains both upstream 5′ (5′-TTCAGGACTAGTCTTTTAGGTCAAAAAGAAGAAGCTTT GTAACCGTTGGTTTCCG-3′; SEQ ID NO:38) and downstream 3′ (5′-CTATCGCT-3′; SEQ ID NO:39) native sequences from the original human tRNA Val coding region (pHtV1). These sequences were retained in the event that they enhanced transcriptional activity. This REC expresses the approximately 74 nt shRNA (5′-GTTTCCGTAGTGTAGTGGTTGAATGGCGTC AAGGTGGACGTTCGACTCTGGTTCACCTTGATGCCGTTCTTTTTCTATCGCT-3′; SEQ ID NO:40) shown in FIG. 24B, which is directed against eGFP.

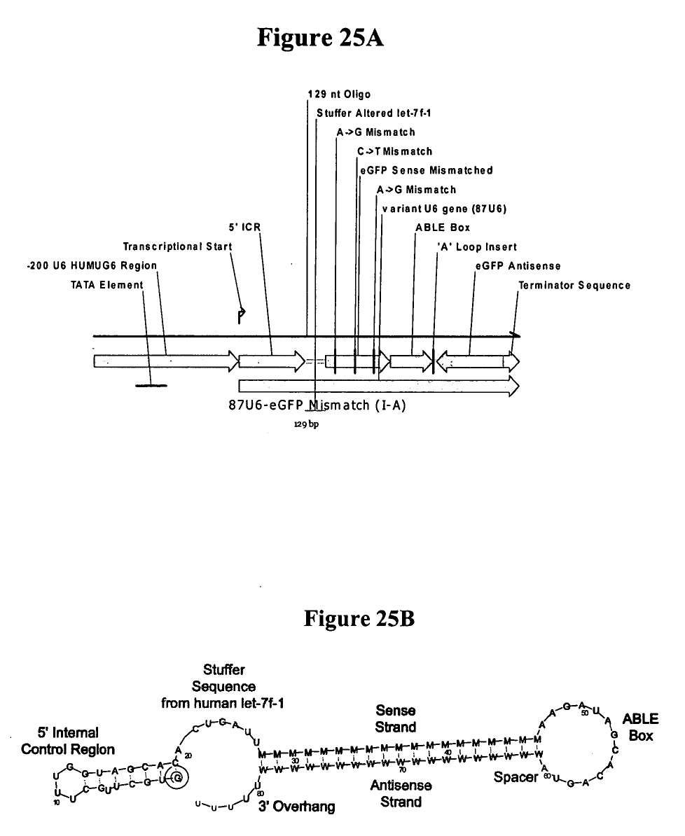

A second REC, labelled as “87U6-eGFP Mismatch (I-A)” in FIG. 25A, is based on the human 87U6 (Genbank accession: HUMUG6; U6 gene variant) internal promoter. The 129 bp sequence (5′-TCGATTTCTTGGCTTTATATATCTTGTGGAAAGGACGAAACA CCGTGCTTGCTTTGGTAGCACACTGATTGCAGGCTGATCCTGAGGTTCAAGATAGCA CAGTAGAACTTCAGGGTCAGCTTGCTTTTT-3′; SEQ ID NO:41) contains a 5′ hairpin comprising the native “5′ Internal Control Region” (5′-GTGCTTGCTTTGGTAGCACA-3′: SEQ ID NO:42) and a loop sequence comprising an “ABLE Box” (5′-AAGATAGCACAGT-3′; SEQ ID NO:43). The sequence contains the native upstream 5′ (5′-TCGATTTCTTGGCTTTATATA TCTTGTGGAAAGGACGAAACACC-3′; SEQ ID NO:44) sequence from the original human 87U6 coding region. This sequence was retained in the event that it enhances transcriptional activity. In addition, a short stuffer sequence has been inserted which maintains proper spacing between the 5′ ICR and ABLE Boxes. This sequence (5′-CTGATT-3′; SEQ ID NO:45) was taken from the human miRNA let-7f-1 gene and may facilitate nuclear export to the cytosol. This REC expresses the approximately 85 nt shRNA (5′-GTGCTTGCTTTGGTAGCACACTG ATTGCAGGCTGATCCTGAGGTTCAAGATAGCACAGTAGAACTTCAGGGTCAGCTTGC TTTTT-3′; SEQ ID NO:46) shown in FIG. 25B, which also is directed against eGFP.

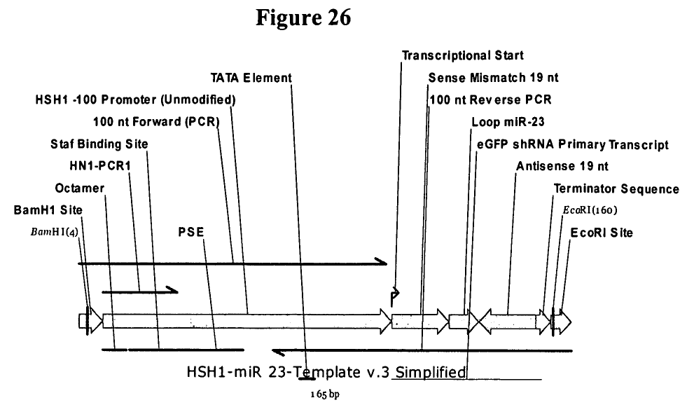

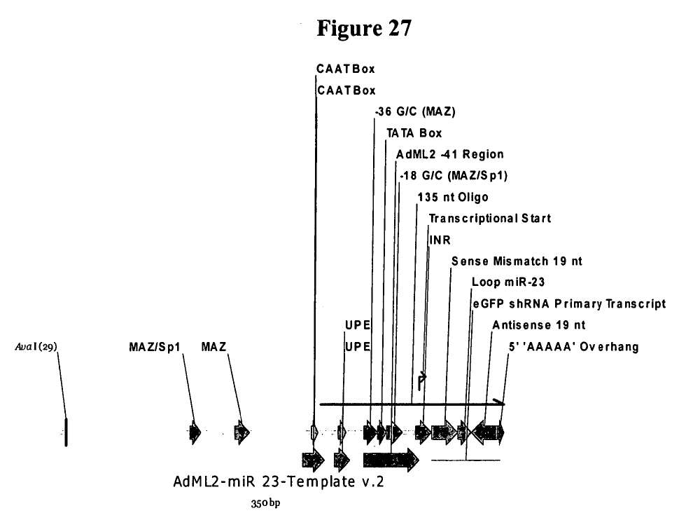

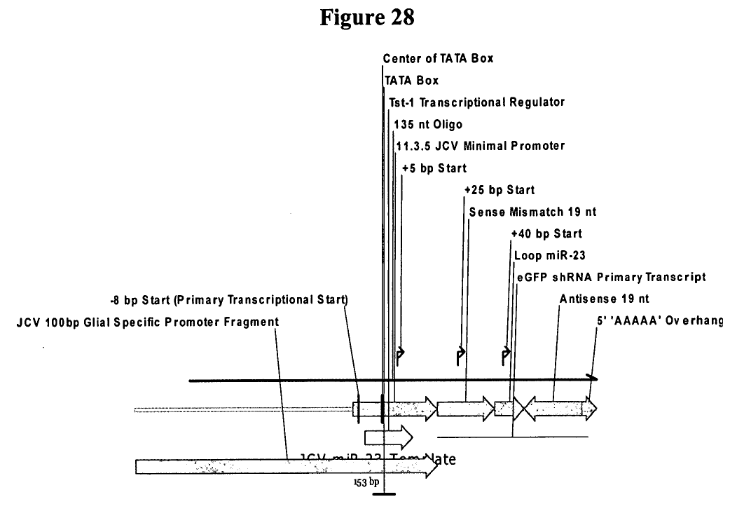

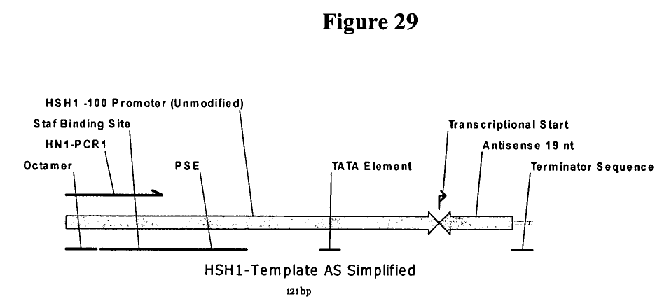

Additional RECs, which embody variations on promoters described above, especially in Examples 1 and 10, are depicted schematically in FIGS. 26-29. The nucleic acid sequences of these RECs are 5′-CGGGATCCATTTGCATGTCGCTATGTGTTCTGGGA AATCACCATAAACGTGAAATGTCTTTGGATTTGGGAATCTTATAAGTTCTGTATGAG ACCACTCTTTCCCNNNNNNNNNNNNNNNNNNCTTCCTGTCANNNNNNNNNNNNN NNNNNTTTTTGAATTCC-3′; SEQ ID NO:47, FIG. 26), 5′-CCCGTATACAGACTTG AGAGGCCTGTCCTCGAGCGGTGTTCCGCGGTCCTCCTCGTATAGAAACTCGGACCAC TCTGAGACGAAGGCTCGCGTCCAGGCCAGCACGAAGGAGGCTAAGTGGGAGGGGTA GCGGTCGTTGTCCACTAGGGGGTCCACTCGCTCCAGGGTGTGAAGACACATGTCGCC CTCTTCGGCATCAAGGAAGGTGATTGGTTTATAGGTGTAGGCCACGTGACCGGGTGT TCCTGAAGGGGGGCTATAAAAGGGGGTGGGGGCGCGTTCGTCCTCACTCTCTTCNNN NNNNNNNNNNNNNNNNCTTCCTGTCANNNNNNNNNNNNNNNNNNNNTTTTT-3′; SEQ ID NO:48, FIG. 27), 5′-TGGCTCCCTAGGTATGAGCTCATGCTTGG CTGGCAGCCATCCAGTTTTAGCCAGCTCCTCCCTACCTTCCCTTTTTTTTATATATAC AGGAGGCCGAGGCNNNNNNNNNNNNNNNNNNCTTCCTGTCANNNNNNNNNNNNN NNNNNNTTTTT-3′; SEQ ID NO:49, FIG. 28), and 5′-ATTTGCATGTCGCTATGTGT TCTGGGAAATCACCATAAACGTGAAATGTCTTTGGATTTGGGAATCTTATAAGTTCT GTATGAGACCACTCTTTCCCNNNNNNNNNNNNNNNNNNTTTTT-3′; SEQ ID NO:50, FIG. 29), wherein the regions denoted by Ns contain the sense and antisense strands of the shRNA transcript to be generated. The REC depicted in FIG. 29 is designed to function by antisense effect, rather than through RNA interference.

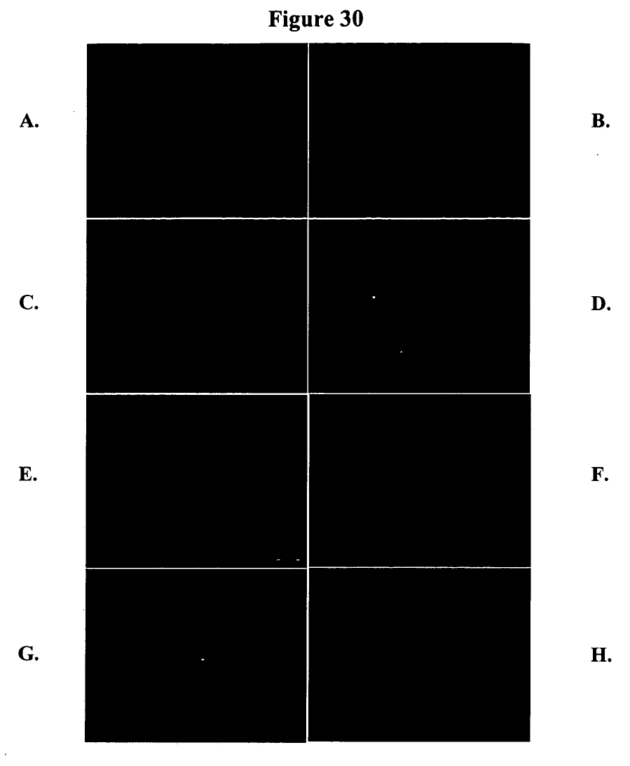

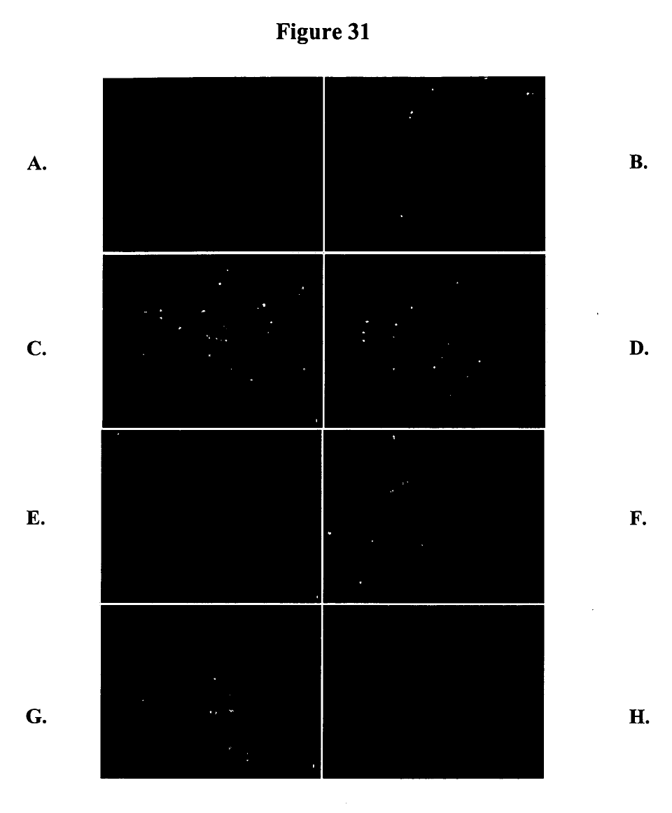

The functionality of two of these RECs, tRNA Val-B Box-eGFP (7) Mismatch (J-A), hereinafter “J-A”, and 87U6-eGFP Mismatch (I-A), hereinafter “I-A,” were confirmed by co-transfecting pCR2.1-TOPO plasmids containing these RECs into cultured human 293 cells along with either a negative control plasmid (pUC19) or an eGFP expression plasmid. The amount of fluorescence was then determined by fluorescent microscopic observation at 48 hr (FIG. 30) or 72 hr (FIG. 31), respectively. At both time points, co-transfection of 200 ng of the eGFP expression plasmid together with a plasmid bearing either the IA or the JA RECs produced a dose-dependent decrease in the number of fluorescent cells. These results were quantified by fluorescence-activated cell sorting. These studies further validate the utility of the compact vector of the instant invention for introducing dsRNA into cells.

### Example 16

Administration of Compact Synthetic Vectors to Express Functional RNA Molecules In Vivo. Having verified that the compact synthetic vectors of the instant invention may be easily constructed and successfully employed to express siRNA molecules in cultured cells, the efficacy of these vectors in vivo may be tested using various animal models. For example, compact synthetic vectors of any of the designs described hereinabove may be generated through the synthesis and annealing of complementary oligonucleotides. These vectors may be linked to appropriate targeting peptides, using for example the coupling method described above in Example 3 or other coupling methods known to those of ordinary skill in the art. The peptide-linked vectors then may be administered either systemically or locally to experimental animals to facilitate delivery of the vector to specific target cells in vivo.

One animal model that may be useful to test the in vivo efficacy of the vectors of the instant invention is the transgenic mouse in which eGFP is expressed under the regulatory control of the β-actin promoter (C57BL/6-TgN(ACTbEGFP)1Osb. This animal is commercially available from Jackson Laboratories (Jackson Labs stock 003291). Compact synthetic vectors containing the RECs described above in Examples 2 or 15, which express siRNA molecules specific for the eGFP gene, could be synthesized, linked to an appropriate targeting peptide, and administered to this transgenic animal. Levels of eGFP-mediated fluorescence in the targeted cells then could be monitored as an indicator of the efficiency and specificity of the RNA interference. Targeted cells could be observed in situ, or harvested for quantitation by fluorescence-activated cell sorting. The artisan of ordinary skill would recognize that many other combinations of RECs, functional RNA molecules, and animal models also may be suitable for in vivo expression of this compact synthetic vector.

The foregoing examples merely illustrate the principles of the invention. Various modifications and alterations to the described embodiments will be apparent to those skilled in the art in view of the teachings herein. It will thus be appreciated that those skilled in the art will be able to devise numerous vectors that, although not explicitly shown or described herein, embody the principles of the invention and are thus within the spirit and scope of the invention.

We claim:

1. A synthetic double-stranded deoxyribonucleic acid (DNA) vector comprising one or more pairs of chemically-synthesized, overlapping complementary oligonucleotides, wherein the vector comprises a ribonucleic acid (RNA) promoter, a region to be transcribed into a RNA molecule, and a transcriptional termination sequence.

2. The vector of claim 1, wherein the vector is linear.

3. The vector of claim 1, wherein the vector is circular.

4. The vector of claim 1, wherein the promoter is selected from the group consisting of human H1 polymerase II promoter, human type 1 polymerase III promoter, human type 2 polymerase III promoter, human type 3 polymerase III promoter, human pol II promoter, adenovirus major late promoter, and tissue-specific or inducible variants thereof.

5. The vector of claim 4, wherein the promoter region has the sequence set forth by SEQ ID NO:20.

6. The vector of claim 4, wherein the promoter region has the sequence set forth by SEQ ID NO:21.

7. The vector of claim 4, wherein the promoter region has the sequence set forth by SEQ ID NO:22.

8. An isolated nucleic acid selected from the group consisting of SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, wherein said nucleic acid is a vector.

9. The vector of claim 4, wherein the tissue-specific variant promoter comprises minimal promoter elements from a gene selected from the group consisting of prepro-endothelin-1 gene, myelin basic protein gene, metallothionein gene, neurofibramatosis-1 gene, growth hormone factor 1 gene, peripherin gene, fibroin gene, JC virus gene, and period-1 gene.

10. The vector of claim 9, wherein the tissue-specific variant promoter has the sequence set forth by SEQ ID NO:7.

11. The vector of claim 4, wherein the inducible variant promoter is the human pol II promoter comprising the estrogen response elements A and B or SEQ ID NO:10 and SEQ ID NO:11, respectively.

12. The vector of claim 4, wherein the pol II promoter further comprises a tethered transactivator peptide.

13. The vector of claim 12, wherein the transactivator peptide is a peptide selected from a group consisting of one or more of peptides comprising the sequence of SEQ ID NO:8 and SEQ ID NO:9.

14. The vector of claim 1, wherein the region to be transcribed is a DNA sequence encoding a ss or ds RNA molecule.

15. The vector of claim 14, wherein the RNA molecule is selected from the group consisting of a hairpin RNA molecule that can be converted into a short, interfering RNA by RNase III, an antisense oligonucleotide, and a ribozyme.

16. The vector of claim 14, wherein the RNA molecule has the sequence of SEQ ID NO:1.

17. The vector of claim 14, wherein the RNA molecule has the sequence of SEQ ID NO:16.

18. The vector of claim 1, further comprising a heteroduplex bubble.

19. The vector of claim 1, wherein the one or more oligonucleotides comprise a covalently attached moiety selected from the group consisting of a protein transduction domain, an RGD peptide, a receptor ligand, an antibody, a nuclear localization sequence, an endosmolytic peptide, a fluorescent beacon, and combinations thereof.

20. The vector of claim 1, wherein the vector is from about 50 bp to about 135 bp in length.

21. The vector of claim 1, wherein the vector is from about 50 bp to about 2000 bp in length.

22. A host cell comprising the vector of claim 1.

23. A method of generating the vector of claim 1 comprising annealing two or more complementary synthetic oligonucleotides to form a double-stranded DNA molecule.

24. The method of claim 23, wherein said oligonucleotides are ligated extracellularly.

25. The method of claim 24, wherein said oligonucleotides are ligated intracellularly.

26. A method for expressing a ss or ds RNA molecule in a target cell comprising administering to the target cell the vector of claim 1 in an amount effective to express a ss or ds RNA molecule in the target cell.

27. A method for expressing a ss or ds RNA molecule in a target cell comprising administering to the target cell the vector of claim 14, wherein the ss or ds RNA molecule is expressed.

28. A method of inhibiting gene expression in a target cell comprising administering to a target cell the vector of claim 1 in an amount effective to inhibit gene expression in the target cell.

29. A method of inhibiting gene expression in a target cell comprising administering to a target cell the vector of claim 14 in an amount effective to inhibit gene expression in the target cell.

30. A synthetic vector made by the method of claim 23.

31. A synthetic vector made by the method of claim 24.

32. A synthetic vector made by the method of claim 25.
